(19) United States
(12) Patent Application Publication
(10) Pub. No.: US 2025/0316386 A1
(43) Pub. Date: Oct. 9, 2025

(54) SYSTEMS AND METHODS OF REINFORCED LEARNING IN NEURONAL CULTURES FOR ASSESSMENT OF COGNITIVE FUNCTIONS ASSOCIATED WITH PSYCHIATRIC DISORDERS (PD) AND PERSONALIZED TREATMENT EVALUATION

(71) Applicant: ITAYANDBIOND LTD, Tel Aviv-Yaffo (IL)

(72) Inventors: Nisim PERETS, Tel Aviv (IL); Shmulik BEZALEL, Limassol (CY); Nir WAISKOPF, Jerusalem (IL); Oren BOUSKILA, Yad Natan (IL); Naresh MUTUKULA, Jerusalem (IL)

(21) Appl. No.: 19/200,006

(22) Filed: May 6, 2025

Related U.S. Application Data
(63) Continuation of application No. PCT/IL2023/051158, filed on Nov. 9, 2023.
(60) Provisional application No. 63/423,892, filed on Nov. 9, 2022.

(51) Int. Cl. G16H 50/20

(52) U.S. Cl. CPC G16H 50/20

(57) ABSTRACT

Disclosed are a method and a system for the assessment of the severity of a psychiatric disorder (PD) using measurements of computational and high-order functions in brain organoid neurons.

### CROSS REFERENCE TO RELATED APPLICATIONS

This application is a Bypass Continuation of PCT Patent Application No. PCT/IL2023/051158 having International filing date of Nov. 9, 2023, which claims the benefit of priority of U.S. Provisional Patent Application No. 63/423,892, filed Nov. 9, 2022, the contents of which are all incorporated herein by reference in their entirety.

### TECHNICAL FIELD

The present disclosure generally relates to evaluation of cognitive functions related to complex, multifactorial psychiatric disorder (PD), and personalized evaluation of treatment efficacy, using reinforcement/conditional learning-driven computer simulation in brain organoid and stem cell-derived 2D neuronal cultures.

### BACKGROUND OF THE INVENTION

Psychiatric disorders (PD) include a range of conditions, including neurologic, neurodevelopmental, and neurodegenerative disorders that affect mental, emotional and/or behavioral aspects in a way that disturbs and impairs the function of an individual.

While the biological mechanism underlying neurologic, neurodevelopmental, and neurodegenerative disorders are versatile, a common dominator for these PD would be the association thereof with perturbed cognitive functionalities, that flawed the mental, emotional and/or behavior capabilities of the patient.

PD, with emphasis on neurodevelopmental disorders such as Attention Deficit Hyperactivity Disorder (ADHD/ADD), Major depression, or Bipolar disorder, deeply impact life quality, notwithstanding the relative manageability of these disorders using psychoactive or anticonvulsant medications.

Other neurodegenerative or neurodevelopmental disorders, such as Autism Spectrum Disorders (ASD) currently lack pharmaceutical solutions to treat the core symptoms.

Perturbed cognitive functionalities associated with PD include, but are not limited to cognitive impairment/rigidity (e.g., adaptive learning), social problems (e.g., communication and social interaction), repetitive and restricted patterns of behavior, motivation, and/or attention, and more.

PD may have genetic bases that may determine the course of development of the disorder (i.e., genetic PD), yet in some manifestations, PD involves a strong influence of other biological non-genetic factors (such as epigenetic) that impact the risk and contribute to the development of the disorder and its symptoms (i.e., non-genetic PD).

The complex etiology and genetic bases of PD especially of non-genetic PD, means that evaluation of PD severity using molecular genetic tools is less feasible, and practically restricts evaluation of the severity of PD to clinical signs relying on symptoms, phenotypic behavior, and disturbances of mood or psychosis. Therefore, currently, the process of evaluation of PD requires the presence, involvement, and preferably the cooperation of the patient through a series of sessions and tasks that may be laborious and exhausting, as well as qualitative and subjective to some extent.

Furthermore, while early diagnosis is critical for behavioral intervention and in the future also for early treatment, currently evaluation of PD is only possible when the subject reaches childhood or later, in some cases only in adolescence.

There is therefore an urgent need for patient-independent evaluation, and more quantitative means to diagnose PD and evaluate its level of severity, as early as possible, preferably during prenatal stages.

Brain organoids are 3D-cultured cell aggregates or self-assembled structures, derived from induced pluripotent stem cells (iPSC) that can recapitulate the structure and function of different brain regions, including high-order brain regions involved in cognition and learning, such as the cerebral cortex.

Brain organoids and stem cell-derived 2D neuronal culture therefore may be especially useful in modeling neural circuits, and conditional and adaptive learning, and for investigating PD related imperfections in cognitive functionalities, in neurologic, neurodevelopmental, and/or neurodegenerative patients.

In vitro lab-grown tissues can provide information on the development and molecular profile of the neurons and other cells, yet it is hard to determine the level of functionality in an in-vitro tissue.

Therefore, there is a need to develop a reliable, functional assay that can provide information on the functionality of the neural network and estimate its ability to encode information properly.

### SUMMARY OF THE INVENTION

According to some aspects, the present disclosure provides systems and methods for the assessment of a behavioral-like response associated with psychiatric disorders (PD), and relies on the basic synaptic abilities to respond to stimuli sessions assayed according to principles of conditional reinforcement learning, applied to brain organoids and neuronal cultures thereof, derived from healthy and PD patients.

Advantageously, the herein disclosed assessment of PD-severity relies on determining the brain organoid response, or the response of stem cell-derived 2D neuronal culture, to stimuli, including electrophysiological stimuli, and is driven by interplay between the brain organoid or the neuronal culture, and components of the system that repeatedly stimulate the brain organoid, or the neuronal culture, in an open loop or closed loop modes, and sense neuronal activity in response to the provided stimuli. This neuro-computational stimuli-response assay determine the organoids' behavior, or the neuronal culture behavior, based on their neural network activity, and classify it according to similarities to predicted behaviors of healthy or PD-derived organoids or stem cell-derived neuronal cultures.

The abovementioned process simulates the functionality of the neural network and its ability to respond and learn, this underlies the ability of the herein provided systems and methods to effectively diagnose PD and assess its severity. Finally, the process can be visually simulated as a computer game representing functional cognitive assay, such as but not limited to social interaction or repetitive behavior.

Further advantageous, the systems and methods provide a diagnostic tool that may be indicative of PD and/or its level of severity, and may also be used as a platform for PD drug discovery and/or for personalized evaluation of treatment efficacy with medicine (i.e., predictive tool for the clinical success of treatment with a medicament) for a psychiatric, neurologic, neurodevelopmental and/or neurodegenerative condition, thereby customizing optimal treatments for PD patients and promoting biomarkers discovery by complemental biochemical evaluations.

According to one aspect, there is provided a system for assessment of a psychiatric disorder (PD), the system comprising:
(i) a brain organoid and/or stem cell-derived 2D neuronal culture;
(ii) a stimuli system capable of delivering stimuli to the brain organoid and/or the neuronal culture; (ii)
(iii) a sensor coupled to a recorder capable of detecting and recording one or more signals indicative of neuronal function/activity of the brain organoid and/or the neuronal culture;
(iv) a micro-controller unit (MCU) configured to receive, integrate and/or transmit data of the one or more signals; and
(v) a computer/processor configured to:
   a. send instructions to the stimuli system to provide one or more stimuli sessions, each session comprising a stimuli provided to the brain organoid and/or the neuronal culture;
   b. obtain data recorded in response to the one or more stimuli sessions, the data indicative of neuronal function/activity of the brain organoid and/or the neuronal culture;
   c. determine a brain-organoid behavior and/or a neuronal culture behavior based on the recorded data; and
   d. apply an AI algorithm on the brain-organoids behavior and/or the neuronal culture behavior to thereby classify the brain organoid based on a degree of similarity of the determined brain-organoid behavior to a predicted behavior of a PD-derived brain organoid and/or a heathy organoid, and/or to thereby classify the neuronal culture based on a degree of similarity of the determined neuronal culture behavior to a predicted behavior of a PD-derived neuronal culture and/or a heathy culture. Each possibility is a separate embodiment.

According to one aspect, there is provided a system for assessment of a psychiatric disorder (PD), the system comprising:
(i) a brain organoid;
(ii) a stimuli system capable of delivering stimuli to the brain organoid;
(iii) a sensor coupled to a recorder capable of detecting and recording one or more signals indicative of neuronal function/activity of the brain organoid;
(iv) a micro-controller unit (MCU) configured to receive, integrate and/or transmit data of the one or more signals; and
(v) a computer/processor configured to:
   a. send instructions to the stimuli system to provide one or more stimuli sessions, each session comprising a stimuli provided to the brain organoid;
   b. obtain from the MCU data recorded in response to the one or more stimuli sessions, the data indicative of neuronal function/activity of the brain organoid;
   c. determine a brain-organoid behavior based on the recorded data; and
   d. apply an AI algorithm on the brain-organoids behavior to thereby classify the brain organoid based on a degree of similarity of the determined brain-organoid behavior to a predicted behavior of a PD-derived brain organoid and/or a heathy organoid. Each possibility is a separate embodiment.

According to some embodiments, the system comprises an open loop, in which the stimulus provided to the brain organoid in the one or more sessions are predetermined.

In some embodiments, the AI algorithm is trained on brain-organoids behaviors of a plurality of healthy and/or PD derived brain organoids in response to the predetermined stimulus, wherein the training data is labeled according to one or more parameters of the stimulus. Each possibility is a separate embodiment. (open loop)

In some related embodiments, the AI algorithm is continuously reinforced, based on the determined brain-organoid behavior, to thereby improve the predicted behavior. (open loop)

According to some embodiments, the system comprises a closed loop, in which the stimulus provided to the brain organoid is determined according to the determined brain-organoid behavior. (closed loop)

In some embodiments, the AI algorithm is trained on brain-organoids behaviors of a plurality of healthy and/or PD derived brain organoids, wherein the training data is labeled according to one or more parameters of the treatment/stimulus. Each possibility is a separate embodiment. (closed loop)

In some specific embodiments, the processor is configured to instruct to the stimuli system to provide at least two sessions, wherein the stimuli provided in a latter session is determined based on the brain-organoids behavior determined in response to one or more former stimuli sessions. (closed loop)

In some further specific embodiments, the stimuli provided in a latter session comprises a positive or negative feedback; and wherein a change in the brain-organoids behavior between a former and the latter sessions is indicative of a learning behavior response of the brain organoid. Each possibility is a separate embodiment. (closed loop)

In some related embodiments, classifying the brain organoid is based on a degree of similarity of the learning-behavior response to a predicted learning-behavior response of a PD-derived brain organoid and/or of a heathy organoid. Each possibility is a separate embodiment. (closed loop)

In some embodiments, the system further comprises a visualization component presenting a visual simulation representative of the determined organoid behavior.

In some specific embodiments, the visual simulation comprises a computer game evaluating cognitive abilities selected from one or more of: memory, cognitive rigidity, motivation, repetitive behavior, attention, social interaction, processing speed, executive function, numerical abilities, and/or facial expression, or any combination thereof. Each possibility is a separate embodiment.

According to some embodiments, the processor is further configured to assess the severity of PD based on the similarity.

In another embodiment, the processor is further configured to repeat steps a-c on the brain organoid after treatment thereof with a neurological, neurodevelopmental and/or neurodegenerative medicament, or any combination thereof. Each possibility is a separate embodiment.

In yet another embodiment, the processor is further configured to repeat steps a-c on a brain organoid obtained from a same subject after neurological neurodevelopmental and/or neurodegenerative treatment of said subject, or any combination thereof. Each possibility is a separate embodiment.

In some specific embodiments, the neurological, neurodevelopmental and/or neurodegenerative treatment comprises a medicament. Each possibility is a separate embodiment.

In some embodiments, the processor is further configured to determine an efficacy of the treatment.

According to some embodiments, the brain organoid is derived from one or more of prenatal cells, neonatal cells, cells of a mature baby, cells of a toddler, cells of a child, cells of a teen, and cells of an adult, or any combination thereof. Each possibility is a separate embodiment.

In some embodiments, the brain organoid is an undetermined brain organoid having unknown severity of PD.

In some embodiment, the obtained brain organoid comprises 3D brain organoid in culture.

In related embodiments, the obtained brain organoid comprises tissue and/or cells thereof in 2D culture, and wherein the tissue and/or cells comprise sliced tissue and/or dissociated cells resulted from any of enzymatic, chemical, and/or mechanical processing of a 3D brain organoid, or any combination thereof. Each possibility is a separate embodiment.

In some embodiments, the sensor comprises one or more multi-array electrodes (MAE) coupled to one or more recording head stage (RHS).

In some embodiments, the stimuli system and the multi-array electrode (MAE) are same or different. Each possibility is a separate embodiment.

In some embodiments, the MCU is connected to a wireless radio transmitter (RF) or a micro transmitter (MT) connecting it to at least one remote MCU.

In some embodiments, the MCU is connected to a processor/computer or is an integral part thereof. Each possibility is a separate embodiment.

In related embodiments, at least the MAE, RHS and a plate holder for culturing the brain organoid are integrated in an all-in-one device.

In further related embodiments, the all-in-one device further comprises one or more of a stimuli system, an MCU and/or a processor, or any combination thereof. Each possibility is a separate embodiment.

According to some embodiments, the one or more signal indicative of the neuronal function/activity of the brain organoid comprises an electrophysiological signal; and wherein the sensor comprises MAE.

According to some embodiments, the one or more signal indicative of the neuronal function/activity of the brain organoid comprises a light signal of an activity reporter; and wherein the sensor comprises an imaging device.

In some embodiments, the data/information indicative of neuronal function/activity of the brain organoid comprises information of long-term measurements.

In some embodiments, the stimuli/treatment provided by stimuli system comprises one or more of electrophysiological stimuli, optic/light stimulus, heat, a chemical agent/drug, or any combination thereof. Each possibility is a separate embodiment.

In some embodiments, the stimuli/treatment provided by the stimuli system comprises electrophysiological stimuli.

According to some embodiments, at least some of the processing is done with a field-programmable gate array (FPGA).

In some embodiments, the data indicative of the neuronal function/activity comprises spatiotemporal propagation including spatial distribution and/or time after stimulation, intensity, frequency, and amplitude of the detected signal, or any combination thereof. Each possibility is a separate embodiment.

In some specific embodiments, the data indicative of the neuronal function/activity comprises spatiotemporal propagation including spatial distribution and/or time after stimulation. Each possibility is a separate embodiment.

In some embodiments, the PD comprises non-genetic PD.

According to some embodiments, the PD is selected from one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

According to a specific embodiment, the PD is Autistic Spectrum Disorder (ASD). According to further specific embodiment, the ASD is non-syndromic idiopathic ASD.

According to another aspect, there is provided method for assessment of a psychiatric disorder (PD), the method comprising:
a. obtaining a brain organoid;
b. providing one or more stimuli sessions, each session comprising a stimuli provided to the brain organoid;
c. obtaining data recorded in response to the one or more treatment/stimuli sessions, the data is indicative of neuronal function/activity of the brain organoid;
d. determining a brain-organoids behavior based on the recorded data; and
e. applying an AI algorithm on the brain-organoids behavior for classifying the brain organoid based on a degree of similarity of the determined brain-organoid behavior to a predicted behavior of a PD-derived brain organoid and/or a heathy organoid.

According to a specific embodiment, the method comprises an open loop, in which the treatment/stimulus provided to the brain organoid in the one or more sessions are predetermined.

In some embodiments, the AI algorithm is trained on brain-organoids behaviors of a plurality of healthy and/or PD derived brain organoids in response to the predetermined treatment/stimulus, wherein the training data is labeled according to one or more predetermined parameters of the treatment/stimulus. Each possibility is a separate embodiment. (open loop)

In a related embodiment, the AI algorithm is continuously reinforced, based on the determined brain-organoid behavior, to thereby improve the predicted behavior. (open loop)

According to a specific embodiment, the method comprises a closed loop, in which the stimulus provided to the brain organoid is determined according to the determined brain-organoid behavior.

In some embodiments, the AI algorithm is a trained on brain-organoids behaviors of a plurality of healthy and/or PD derived brain organoids, wherein the training data is labeled according to changes in one or more parameters of the treatment/stimulus. (closed loop)

In some embodiments, the method comprises at least two sessions, wherein the stimuli provided in a latter session is determined based on the brain-organoids behavior determined in response to one or more former stimuli sessions. (closed loop)

In some specific embodiments, the stimuli provided in a latter session comprises a positive or negative feedback; and wherein a change in the brain-organoids behavior between a former and the latter sessions is indicative of a learning behavior response of the brain organoid. Each possibility is a separate embodiment. (closed loop)

In some embodiments, classifying the brain organoid is based on a degree of similarity of the learning-behavior response to a predicted learning-behavior response of a PD-derived brain organoid and/or of a heathy organoid. Each possibility is a separate embodiment. (closed loop)

In some embodiments, the method further comprises generating a visual simulation representative of the determined organoid behavior.

In some related embodiments, the visual simulation comprises a computer game configured to evaluate one or more of: memory, cognitive rigidity, motivation, repetitive behavior, attention, social interaction, processing speed, executive function, numerical abilities, and/or facial expression, or any combination thereof. Each possibility is a separate embodiment.

In some embodiments, the method further comprises assessing the severity of PD based on the similarity.

In some embodiments, the method further comprising repeating steps b-d on the brain organoid after treatment thereof with a psychiatric, neurodevelopmental and/or neurological medicament, or any combination thereof. Each possibility is a separate embodiment.

In some embodiments, the method further comprising repeating steps b-d on a brain organoid obtained from a same subject after treatment of said subject with a neurological, neurodevelopmental and/or neurodegenerative medicament, or any combination thereof. Each possibility is a separate embodiment.

In some embodiments, the method further comprising determining an efficacy of the treatment.

In some embodiments, the PD comprises non-genetic PD.

According to some embodiments, the PD comprises one or more neurological, neurodevelopmental and/or neurodegenerative condition, or any combination thereof. Each possibility is a separate embodiment.

In some embodiments the PD is selected from one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

According to yet another aspect, there is provided a method for training an AI algorithm for determining organoids behavior, the method comprising:
a. obtaining a plurality of PD-derived brain organoid and a plurality of healthy brain organoids;
b. providing one or more stimuli session(s), each session comprising stimuli provided to the brain organoid;
c. obtaining data recorded in response to the one or more treatment/stimuli session(s), the data is indicative of neuronal function/activity of the brain organoid;
d. labeling the data according to parameters of the one or more stimuli sessions and associating the labeled data with the PD-derived brain organoid and/or with the plurality of healthy brain organoid;
e. applying an AI algorithm on the data to learn patterns and relationships and to adjust parameters of a model for organoid behavior prediction; thereby training the algorithm for determining a brain-organoids behavior based on the data recorded in response to the one or more treatment/stimuli session(s).

According to some embodiments, the AI algorithm is further trained to classify the organoids plurality of PD-derived brain organoids and/or healthy organoids based on the determined organoids' behavior as having ‘PD-derived behavior’ or a ‘heathy behavior’; thereby classifying the brain organoids based on a degree of similarity of their determined behavior to a predicted behavior of a PD-derived brain organoid and/or a heathy organoid. Each possibility is a separate embodiment.

In some embodiments, the obtaining of PD-derived brain organoid comprises organoids having a range of PD severities, and wherein the association of the labeled data with the PD-derived brain organoid comprises associating the labeled data with the range of PD severities; thereby augmenting the prediction behavior model to include a range of severities.

In another embodiment, the data indicative of neuronal function/activity of the brain organoid is divided to a ‘training dataset’ and ‘validation set’, and wherein the ‘validation set’ comprises unlabeled data used to improve model performance.

In some embodiments, the data indicative of the neuronal function/activity comprises spatiotemporal propagation including spatial distribution and time after stimulation, intensity, frequency, and amplitude of the detected signal, or any combination thereof. Each possibility is a separate embodiment.

In a specific embodiment, the data indicative of the neuronal function/activity comprises spatiotemporal propagation including spatial distribution and time after stimulation.

In some embodiments, the AI algorithm is selected from one or more of supervised learning, unsupervised learning, semi-supervised learning, reinforced learning, self-supervised learning, transfer learning, meta-learning, evolutionary algorithms, or any combination thereof. Each possibility is a separate embodiment.

In some embodiments, the AI algorithm is a supervised machine learning algorithm capable of regression and/or classification selected from one or more of Support-vector machines, Linear regression, Logistic regression, Random Forest, Naive Bayes, Linear discriminant analysis, Decision trees, K-nearest neighbor algorithm, Deep Neural networks, Neural networks (Multilayer perceptron), Gradient Boosting Algorithms, Linear Discriminant Analysis, Ridge Regression and Lasso Regression, Elastic Net, Bayesian Regression, Multiclass Classification Algorithms, Similarity learning, or any combination thereof. Each possibility is a separate embodiment.

According to an embodiment, the method of training comprises an open loop training, wherein the treatments/stimuli provided to the brain organoid in the one or more sessions are predetermined.

According to an embodiment, the method of training comprises closed loop training mode, wherein the treatments/stimuli provided to the brain organoid in the one or more sessions is determined according to the determined brain-organoid behavior.

According to some embodiments, the method of training comprises the stimuli provided to the brain organoid comprises one or more of an electrophysiological stimulus, a heat stimulus, a light stimulus, and a drug, or any combination thereof.

In a specific embodiment, the stimuli provided to the brain organoid comprises an electrophysiological stimulus.

According to some embodiments, the brain organoid is derived from one or more of prenatal cells, neonatal cells, cells of a mature baby, cells of a toddler, cells of a child, cells of a teen, and cells of an adult, or any combination thereof. Each possibility is a separate embodiment.

According to some aspects, there is provided a system for assessment of a learning-behavior response associated with a psychiatric disorder (PD);

In some embodiments, the system comprises a brain organoid;

In some embodiments, the system comprises a stimuli/manipulation system capable of delivering treatment to the brain organoid;

In some embodiments, the system comprises a sensor coupled to a recorder capable of detecting and archiving/recording one or more signals of the brain organoid;

In some embodiments, the system comprises at least one micro-controller unit (MCU) configured to receive, integrate and/or transmit information/data indicative of neuronal function/activity derived from the one or more signals;

In some embodiments, the system comprises a computer/processor connected to and/or running a simulator and configured;

In some embodiments, the system comprises a computer/processor connected to and/or running a simulator and configured to obtain input data comprising the information indicative of neuronal function/activity from the brain organoid;

In some embodiments, the system comprises a computer/processor connected to and/or running a simulator and configured to apply an algorithm to generate a simulation of the brain-organoids behavior;

In some embodiments, the system comprises a computer/processor connected to and/or running a simulator and configured to determine a positive or negative feedback treatment based on the simulated behavior;

In some embodiments, the system comprises a computer/processor connected to and/or running a simulator and configured to send instructions to stimuli/manipulation system to execute the determined positive or negative feedback treatment;

In some embodiments, the system comprises a computer/processor connected to and/or running a simulator and configured to obtain post-treatment input data from the brain organoid comprising information indicative of neuronal function/activity in response to the execution of the determined positive or negative feedback treatment;

In some embodiments, the system comprises a computer/processor connected to and/or running a simulator and configured to apply the algorithm to simulate a post-treatment behavior of the brain organoid data;

In some embodiments, the system comprises a computer/processor connected to and/or running a simulator and configured to repeating steps (c) to (f) X times; wherein X is an integer between 1 and 10000;

In some embodiments, the system comprises a computer/processor connected to and/or running a simulator and configured to compute an output score indicative of the learning-behavior response based on a change in the behavior of the brain organoid;

In some embodiments, the system comprises a computer/processor connected to and/or running a simulator and configured to assess the severity of PD based on similarity to a predetermined learning-behavior response of a PD-derived brain organoid and/or predetermined learning-behavior response of a healthy brain organoid.

In some embodiments, the PD comprises non-genetic PD.

In some embodiments, the brain organoid comprises undetermined brain organoid and/or PD-derived brain organoid.

In some embodiments, determining the feedback and/or computing the output score further comprises taking into consideration a predetermined change in the learning-behavior response of a PD-derived brain organoid and/or a healthy brain organoid.

In some embodiments, the brain organoid is in culture. In some embodiments, the obtained brain organoid comprises 3D brain organoid in culture.

In some embodiments, the obtained brain organoid comprises tissue and/or cells thereof in 2D culture; and wherein the tissue and/or cells comprise sliced tissue and/or dissociated cells resulted from any of enzymatic, chemical, and/or mechanical processing of a 3D brain organoid. Each possibility is a separate embodiment.

In some embodiments, the system is used for drug screening and/or evaluation of efficacy of treatment with a drug applied directly to the brain organoid in culture; wherein the drug comprises one or more of a potential compound/molecule/drug for treating PD or a psychiatric/neurologic drug already in medical use for treating PD. Each possibility is a separate embodiment.

In some embodiments, the processor is further configured to obtain and take under consideration input data from the brain organoid before and after being treated with the drug directly in culture.

In some embodiments, the system is further configured to compute a drug/treatment efficiency score based on a change in the learning-behavior response.

In some embodiments, the treatment delivered by the stimuli/manipulation system to the brain organoid is one or more of an electrical pulse, optic/light stimulus, heat, a chemical agent/drug, or any combination thereof.

In some embodiments, the sensor coupled to a recorder capable of detecting and archiving/recording the one or more signals comprises a multi-array electrode (MAE) coupled to one or more recording head stage (RHS).

In some embodiments, the stimuli/manipulation system capable of delivering electric pulse and the sensor comprises a multi-array electrode (MAE) are same or different.

In some embodiments, the sensor coupled to a recorder capable of detecting and archiving/recording one or more signals comprises an imaging device coupled to a camera.

In some embodiments, the at least one signal detected and archived is an electric signal or an optic/light signal.

In some embodiments, the optic/light signal detected is omitted from a genetic reporter.

In some embodiments, the at least one signal detected and archived comprises information indicative of the neuronal function/activity of the brain organoid.

In some embodiments, the at least one signal detected and archived comprises information of long-term measurements.

In some embodiments, the information indicative of the neuronal function/activity is transferred from the sensor coupled to a recorder to the at least one MCU.

In some embodiments, the MCU is connected to a wireless radio transmitter (RF) or a micro transmitter (MT) connecting it to at least one remote MCU.

In some embodiments, the MCU is connected to a processor/computer or is an integral part thereof; and wherein the processor/computer or at least some of the processing is done with a field-programmable gate array.

In some embodiments, the computer/processor is further connected to same or different stimuli/manipulation system capable of delivering positive or negative feedback treatment to the brain organoid in culture.

In some embodiments, the input data and/or post-treatment input comprises information indicative of the neuronal function/activity of the brain organoids in culture before and/or after treatment.

In some embodiments, the information indicative of the neuronal function/activity comprises duration, intensity, frequency and/or amplitude of the detected signal.

In some embodiments, the information indicative of the neuronal function/activity further comprises spatial information.

In some embodiments, the post-treatment input data comprises information indicative of parameters of the determined positive or negative feedback treatment delivered by the stimuli/manipulation system to the brain organoids in culture.

In some embodiments, the information indicative of parameters of the determined positive or negative treatment delivered by the stimuli/manipulation system to the brain organoids in culture comprises information about concentration, temperature, duration, intensity, frequency and/or amplitude of the stimuli.

In some embodiments, the simulator/visual component comprises one or more of a computer, a computer display, a mouse, a cursor, an artificial or prosthetic limb, a robot, or robotic device.

In some embodiments, the simulated behavior comprises information indicative of the neuronal function/activity

In some embodiments, the simulated behavior comprises psychiatric-related computer games evaluating social interaction, repetitive behavior, cognitive rigidity, and/or face recognition.

In some embodiments, the psychiatric-related computer games comprise positive or negative feedback.

In some embodiments, the determined positive or negative feedback treatment delivered to the brain organoid in culture by the stimuli/manipulation system comprises electrical pulse, optic/light stimulus, heat, a chemical agent/drug, or any combination thereof.

In some embodiments, the PD comprises Autism Spectrum Disorders (ASD), Bipolar disorder, Attention Deficit Hyperactivity Disorder (ADHD/ADD), Schizophrenia, Major Depression, Obsessive-Compulsive Disorders (OCD), Epilepsy, or any combination thereof.

In some embodiments, the psychiatric disorder comprises Autistic Spectrum Disorder (ASD)

In some embodiments, the ASD comprises non-syndromic idiopathic ASD.

According to some aspects, there is provided a method for assessment of a learning-behavior response associated with a psychiatric disorder (PD);

In some embodiments, the method comprises obtaining a brain organoid;

In some embodiments, the method comprises obtaining one or more signals of the brain organoid, wherein the obtaining of one or more signals comprises obtaining information indicative of neuronal function/activity derived from the one or more signals;

In some embodiments, the method comprises computing/processing input data comprising the obtained information indicative of neuronal function/activity from the brain organoid; and generating a computer simulation of the brain-organoid behavior;

In some embodiments, the method comprises determining a positive or negative feedback treatment based on the simulated behavior;

In some embodiments, the method comprises executing/delivering the determined positive or negative feedback treatment to the brain organoid using a stimuli/manipulation system;

In some embodiments, the method comprises obtaining post-treatment input data from the brain organoid comprising information indicative of neuronal function/activity in response to the execution of the determined positive or negative feedback treatment;

In some embodiments, the method comprises simulating a post-treatment behavior of the brain organoid data;

In some embodiments, the method comprises repeating steps (iv) to (vii) X times; and

In some embodiments, the method comprises computing a degree of similarity of the computed learning-behavior response to a predetermined PD-derived learning-behavior response profile and/or to a predetermined healthy-derived learning-behavior response profile and providing an output score indicative of the learning-behavior response based on the degree of similarity thereby assessing the severity of PD.

In some embodiments, the PD comprises non-genetic PD.

In some embodiments, the brain organoid comprises undetermined brain organoid and/or PD-derived brain organoid.

According to some aspects, there is provided method for training a machine learning algorithm for assessment of a learning-behavior response associated with a psychiatric disorder (PD);

In some embodiments, the method for training a machine learning algorithm comprises obtaining information indicative of neuronal function/activity from a plurality of PD-derived brain organoid and a plurality of healthy brain organoid;

In some embodiments, the method for training a machine learning algorithm comprises labeling the information indicative of neuronal function/activity of the plurality of PD-derived brain organoids as ‘PD-derived’ input data and the information indicative of neuronal function/activity of the plurality of healthy brain organoids as ‘healthy-derived’ input data;

In some embodiments, the method for training a machine learning algorithm comprises computing/processing the input data comprising the labeled information; and generating a computer simulation indicative of a behavior of each of the PD-derived brain-organoids and a simulation indicative of a behavior of each of the healthy brain organoids;

In some embodiments, the method for training a machine learning algorithm comprises providing a positive or negative feedback treatment to each of the PD derived and healthy organoids, wherein the treatment is responsive to the simulated behavior; and obtaining post-treatment information indicative of neuronal function/activity of the plurality of PD-derived brain organoids and post-treatment information indicative of neuronal function/activity of the plurality of healthy brain organoids, in response to the provided feedback treatment;

In some embodiments, the method for training a machine learning algorithm comprises labeling the plurality of post-treatment information indicative of neuronal function/activity of the plurality of PD-derived brain organoids as ‘PD-derived post-treatment’ input data and the plurality of information indicative of neuronal function/activity of the plurality of healthy brain organoids as ‘healthy-derived post-treatment’ input data;

In some embodiments, the method for training a machine learning algorithm comprises computing/processing the plurality of post-treatment input data comprising the labeled information; and simulate the behavior of the plurality of PD-derived brain-organoids and the behavior of the plurality of healthy brain organoid;

In some embodiments, the method for training a machine learning algorithm comprises computing a learning behavior response for each of the PD-derived brain-organoids and each of the healthy derived brain organoids, based on a change in the simulated behavior of the organoids in response to the treatment;

In some embodiments, the method for training a machine learning algorithm comprises computing an PD-derived brain organoid learning behavior response profile and a healthy organoid learning behavior response profile based on the learning behavior responses computed for each of the PD-derived and healthy-derived organoids.

In some embodiments, the positive or negative feedback treatment is same or different for the PD-derived brain organoid and the healthy brain organoid.

In some embodiments, each one of the simulations comprises random movement of two dots in 2D space.

In some embodiments, the determining a positive or negative feedback treatment based on the simulated behavior comprises positive feedback when the dots come close together and negative feedback when the dots go apart from each other.

In some embodiments, the method for training a machine learning algorithm further comprising validating the trained algorithm on a validation set comprising a plurality of unlabeled PD-derived behavior simulations and a plurality of unlabeled healthy-derived behavior simulations obtained before and after feedback treatment.

Certain embodiments of the present disclosure may include some, all, or none of the above advantages. One or more technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures.

FIGS. 1A-1B present the system for assessment of a psychiatric disorder (PD).

FIG. 1A schematic illustration of system components (dashed boxes), their structural and functional relations and workflow of the process of determining severity of PD and classifying a brain organoid, or stem cell-derived 2D neuronal culture, based on the response of the neuronal network/behavior of the organoid, or of the stem cell-derived 2D neuronal culture, in response to a predetermined treatment (Open loop), or to positive or negative feedback treatment (Closed loop). The system includes: (1) a brain organoid or stem cell-derived 2D neuronal culture; (2) a sensor (multi-array electrode (MAE)) coupled to a recorder (recording head stage (RHS)) capable of detecting and recording signals from the brain organoid or from the stem cell-derived 2D neuronal culture; (3) a micro-controller unit (MCU) configured to receive, integrate and/or transmit information/data indicative of neuronal function/activity derived from the signals; (4) Optionally FPGA (Field-Programmable Gate Array) or Equivalent Element, before, after or integrated in the MCU, responsible for high-speed parallel signal processing tasks, such as spike detection and classification, as well as the synchronization of stimulation triggers and/or a computer/processor capable of determine response to stimuli/treatment sessions (or determine an organoid behavior, or stem cell-derived 2D neuronal culture, in response to stimuli/treatment sessions), and give instructions to provide a predetermined treatment (Open loop), or a feedback treatment (Closed loop), and computing an output at the end of the sessions; (5) Optionally, a visualization component such as a screen/monitor/robot, or the like, connected to the processor/computer, and capable of presenting the computational simulation (i.e., the stimuli-response sessions/simulation) in a visual manner; (6) a stimuli/manipulation system connected to the processor/computer and capable of delivering treatment to the brain organoid in culture, or to the stem cell-derived 2D neuronal culture. (7) The system computes an output including computation of the overall responses (Open loop), or a learning response (Closed loop), for PD, healthy or undetermined brain organoids, or neuronal culture derived from any developmental stage (e.g., prenatal, neonatal, mature baby, or adult); and (8) assesses probability of severity of PD based on similarity between the neuronal network response and a predicted response, and classify accordingly (9) thereby providing a platform that can be utilized for drug screening and/or as a mean for personalized medicine aiming at evaluation/prediction of clinical success of treatment with a psychiatric drug, including a medicament for neurologic, neurodevelopmental, and neurodegenerative disease.

FIG. 1B schematic illustration of the same system components of FIG. 1A, and relation between them, in basic and scalable formats. The upscaled structure includes for some of the components a plurality of units, including a plurality of cultures brain organoids, plurality of RHS, plurality of MEA, and plurality of monitors.

FIGS. 1C-1D shows a picture presenting a device customized according to the design principles of the system for assessment of PD presented in FIGS. 1A-1B. The device is an ‘organ-on-a chip’ designed to facilitate electrophysiological recordings and electrical stimulation of a biological sample. Shown are MEA positioning area, electrode connector pins, reference and ground pins, amplifying head stage, and 16 pin omnetics connector (FIG. 1C). Also shown are gold-plated pogo pins, removable connection bridges, amplifying head stage, and 16 pin omnetics connector, a grounding cable, and a copper foil cover for sealing of a plate with a 3D brain organoid or with stem cell-derived 2D neuronal culture (FIG. 1D).

FIG. 1E shows a picture presenting a non-limiting example of the system for assessment of PD customized according to the design principles of the system presented in FIGS. 1A-1B. The system components include: (I) the customized device of FIGS. 1C-1D; including a cultured brain organoid, wired to: (II) a stimuli/manipulation system capable of providing an electrophysiological stimulus to the brain organoid in culture, recording signals from the brain organoid, and transmit information/data indicative of neuronal function/activity derived from the signals directly to the computer; (III) a computer/processor capable of communicating with the stimuli/manipulation system.

FIG. 1F shows an enlarged picture of the custom device of FIG. 1E (I). Shown are (1) place holder for MEA to be used as an organ-on-a chip interface for culturing brain organoid, or stem cell-derived 2D neuronal culture (2) a printed circuit board (PCB) (3) conductive contact pin, RHS units and connectors (4) Connectors for Ref and grounding.

FIGS. 2A-2B shows a microscope picture presenting the interface between a brain organoid and the multi electrode array (MEA) belonging to the customized device of FIGS. 1C-1D. The brain organoid is a PD-derived cortical brain organoid (90 days after the beginning of the protocol for preparation of cortical organoids from hiPSCs) generated from cells of an ASD patient. The MEA has 59 ‘channels’ and is capable of sending electrophysiological stimuli in a spatiotemporal controlled manner by activating a sub-set of electrodes according to a desired pattern and parameters.

FIG. 2A shows an image presenting the interface between a 3D brain organoid and the MEA at its on/off states, wherein in the ‘off’ state no electrophysiological stimuli are provided to the 3D brain organoid (Right) and in the ‘on’ state 3 electrodes are activated (Left; red dots). The activation provides an electrophysiological stimulus to the PD-derived 3D brain organoid in a spatiotemporal controlled manner including specific patterns and parameters.

FIG. 2B shows an image presenting the interface between a dissociated brain organoid in 2D culture and the MEA. The PD-derived cells in the culture were dissociated from the brain organoid of FIG. 2A. After its formation (126 days after the beginning of the protocol for preparation of cortical organoids from hiPSCs) the 3D brain organoid was processed/enzymatically digested to dissociated cells, with a commercial Papain dissociation system, which were plated in 2D culture.

FIGS. 3A-3D show microscope images presenting green fluorescence detected from brain organoids in culture (calcium imaging) 14 days after infection thereof with AAV encoding a genetic calcium indicator (GCaMP). Images were taken at 10 fps, 20× NA 0.9 dry objective using Nikon (Yokogawa) spinning disk confocal microscope equipped with an incubator chamber.

FIG. 3A presents dissociated cortical brain organoid (day 126 with respect to the beginning of the protocol for preparation of cortical organoids from hiPSCs) expressing GCaMP8m calcium indicator (AAV9:hSyn1-GCaMP8m).

FIG. 3B presents a small (approx. 0.5 mm wide) clump of organoid tissue from a partially dissociated organoid expressing GCaMP8m calcium indicator (AAV9:hSyn1-GCaMP8m).

FIG. 3C presents electrical activity through calcium imaging recording from a PD-derived cortical brain organoid (Day86 with respect to the beginning of the protocol for preparation of cortical organoids from hiPSCs) generated from cells of an ASD patient expressing GCaMP8m calcium indicator (AAV9: hSyn-jGCaMP7f). White circles mark regions of interest (ROI).

FIG. 3D presents quantification of fluorescence traces recording of ROI from the calcium imaging of FIG. 3C.

FIGS. 4A-4C shows an illustration of computational simulations of brain organoid behavior or stem cell-derived 2D neuronal culture. The simulations are illustrated as computer games representing functional cognition assays being performed by the brain organoid or the stem cell-derived 2D neuronal culture as a ‘player’. The simulation performs assessment of PD severity and/or provides a likelihood of being classified as healthy or PD.

FIG. 4A presents a snapshot of a computer game simulation representing functional cognitive assessment of healthy and PD-like stem cell-derived 2D neuronal culture, as an example for the type of game that can be used to simulate behavior associated with an open loop mode. A predetermined stimuli to the left or right side of the organoid (a golden coin) is ‘answered’ with a behavior response of the ‘player’ that moves (left or right) in a more stimuli-dependent manner as expected from a healthy stem cell-derived 2D neuronal culture or in a more random manner as would be expected from a PD-derived stem cell-derived 2D neuronal culture.

FIG. 4B presents a snapshot of a computer game simulation of a dot moving in 2D space representing functional cognitive assessment of repetitive behavior of healthy and ASD-derived organoid, as an example for the type of game that can be used to simulate behavior associated with an open or closed loop mode. The movement represents an organoid behavior in response to predetermined stimuli, whereas a dot that moves in a periodically manner through space (i.e., in the same pattern) is classified as ASD-derived, while a dot that moves more randomly through space is classified as healthy.

FIG. 4C presents a snapshot of a computer game simulation of two dots moving in 2D space representing functional cognitive assessment of social interaction of healthy and PD-derived brain organoids, as an example of the type of game that can be used to simulate learning-behavior associated with a closed loop mode. The simulation includes a random movement of two dots in 2D space, whereas when the dots come closer to each other interaction occurs and a positive feedback treatment is determined/executed, but when the dots move apart from each other and there is no interaction a negative feedback treatment is determined/executed.

FIG. 4D shows an illustration exemplifying the process determining of a brain-organoids behavior, based on electrical activity data recorded in response to one or more treatment/stimuli session(s). Once electrophysiological raw data (or reporter activity data) is recorded in response to a stimuli session, a response pattern and pattern analysis are performed to learn about the neural network response. In each cycle, stimuli are provided through specific electrodes and the collected activity data from each electrode is filtered and reduced to record only the time in which spikes were detected (Left). Then, in following sessions, multiple repetitions of this stimulation-recording cycle are performed. The mean response pattern is computed taking into account the spike channel (which electrodes detected the signal, i.e., distribution) and the time since the stimulation was plotted (i.e., time after stimuli) (Right). This response pattern is then taken to pattern analysis that compares it to patterns/predicted patterns from healthy and PD samples. Hence determining a behavior includes at least a spatiotemporal analysis of spike propagation.

FIG. 5A: shows representative bright field micrograph images presenting ‘an overview’ of the main stages of generating cortical brain organoids from primary cells. Apparent morphological differences make it clear to distinguish between primary human amniotic epithelial cells (HAEpiCs) (I.), induced pluripotent stem cells (iPSCs) of an established line derived from the HAEpiCs by cellular reprogramming (HAEpiC-iPSC line) (II.), and a 6 day old cortical organoid generated from cells of the HAEpiC-iPSC line (III.).

FIG. 5B: shows representative bright field micrograph images presenting a population of about 15 3D cortical brain organoids at day 130 generated from the HAEpiC-iPSC line, visible to the naked eye in a 6-well culture plate.

FIG. 5C shows representative fluorescence images presenting immunostainings of undifferentiated cells of the HAEpiC-iPSC line for expression pluripotency markers SOX2, NANOG, OCT3/4, as well as Hoechst nuclear staining. Scale bar: 100 μm.

FIG. 5D shows representative fluorescence images presenting immunostainings of a 42 days old HAEpiC-Cortical Organoid for expression of nuclear staining (Hoechst), neuronal marker (TUJ1), neural stem cell markers (SOX2), as well a merged image showing the overlap in their expression pattern presented at a scale of 350 μm (Top; I.), and zoomed in images of Enlarged Neural Vesicle/rosette presented at a scale of 50 μm (Bottom; II.). The enlarged image of a neural vesicle/rosette display neural stem cells (SOX2) around the ventricle and neurons (TUJ1) surrounding the neural stem cells.

FIG. 5E shows representative fluorescence images presenting immunostainings of two 130 days old HAEpiC-Cortical Organoids (Top; I. and Bottom; II.)), for expression of nuclear staining (Hoechst), neuronal marker (TUJ1), neural stem cell markers (SOX2), as well a merged image showing the overlap in their expression pattern presented at a scale of 500 μm.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

As used herein, the singular forms “a”, “an”, and “the” are intended to include the plural forms as well, unless the context clearly indicates otherwise. For example, referring to a psychiatric disorder (PD) may include more than a single PD, and when a reference is made to the brain organoid it may include a reference to multiple brain organoids.

As used herein, the term “about” when referring to a measurable value such as an amount, a temporal duration, and the like, is meant to encompass variations of +20% or in some instances ±10%, or in some instances ±5%, or in some instances ±1%, or in some instances ±0.1% from the specified value, as such variations are appropriate to perform the disclosed methods.

As used herein, the term “comprising” is synonymous with the terms “including,” “containing,” or “characterized by,” and is inclusive or open-ended i.e. does not exclude additional, unrecited elements. The term comprising may be replaced with the term “essentially consisting of” which does not exclude additional, unrecited element, step, or ingredient not specified and which does not affect the basic and novel characteristics of the claimed invention. The term comprising may be replaced with the term “consisting of” which excludes any element, step, or ingredient not specified. In some embodiments, the term essentially consists of, or consisting of, may replace the term comprising.

As used herein, the term “plurality” may refer to brain organoids, and include according to some embodiments, a quantity of more than 5, more than 25, more than 50, more than 100, more than 250, more than 500, or more than 1000 brains. Each possibility is a separate embodiment.

The term “assessing/assessment”, refers to diagnosis PD, i.e., classification of a brain organoid, or stem cell-derived 2D neuronal culture, as PD or healthy, and further evaluation of the severity of PD. This is achieved by comparing the similarity of the determined behavior to predicted behaviors of plurality of healthy or PD-derived organoids or stem cell-derived 2D neuronal cultures. The assessment includes the organoids neural network response, or determining a behavior based on the neural network response to treatment/stimuli provided to the organoid or to the stem cell-derived 2D neuronal culture, during one or more stimuli sessions.

The systems and methods provide a platform for evaluation of PD based on an interactive process between neuronal network, and components of the system in a closed loop or open loop modes, and including a source of stimuli, a sensor, and a processor that is configured to execute neuro-computational simulations that include providing stimuli and determining responses. in an iterative process of reinforcement learning in neurons of the brain organoid, or of stem cell-derived 2D neuronal culture that ultimately encompass a measure for the computational and high-order functions of the neuronal network.

For example, in the closed loop mode, this may be achieved by repeatedly determining positive or negative feedback (i.e., neural behavior/function) based on computer-simulated information indicative of neuronal function/activity in response to preceding feedback, computing the change in response (i.e., the process of reinforcement of learning), and scoring it according to predicted response determined by AI algorithm trained in a similar process of determining learning-behavior response performed on a PD-derived neuronal networks and/or a healthy neuronal network, thereby evaluating the severity of PD.

Therefore, the assessment of PD using the closed loop is performed by determining a learning-behavior response driven/mediated by the interplay between a brain organoid and components of the system that repeatedly stimulate and simulate it.

As used herein the term, “behavior”, is related to the term “neuronal function/activity” and may refer to the response of the neural network or to patterns or relationships that underly the neural network response to stimuli. The behavior response is characteristic of the function/activity of the brain organoid, and stem cell-derived 2D neuronal culture, with respect to those specific stimuli they were exposed to. The behavior/response refers to the data/information that was recorded in response to the stimuli provided during one or mor stimuli sessions.

The behavior refers to a behavior/response determined in response to a process driven/mediated by the open or the closed loop mode.

In some embodiments, the behavior includes a learning-behavior (closed loop).

In some embodiments, brain organoid behavior includes data/information indicative of neuronal function/activity of the brain organoid.

The terms “behavior”, “behavior-like”, “response” and “behavior response”, “neuronal function/activity/response” and “simulated behavior” may be used interchangeably.

As used herein the term, “determined behavior”, may refer to the determined response of the neural network or to patterns or relationships that underly the neural network response to stimuli. Reference is made to the method of determining brain organoid behavior. The determined behavior response is characteristic of the function/activity of the brain organoid, and stem cell-derived 2D neuronal culture, with respect to those specific stimuli they were exposed to, but it may reflect, a reduction in the data/information that was recorded, into a form where it can be classified as ‘PD-derived behavior’ or ‘healthy behavior’, or according to “PD-severity”.

In some embodiments, the learning of the patterns and relationships includes one or more of spatiotemporal propagation including duration or distribution of the signal, intensity, frequency, and amplitude, or any combination thereof.

In some embodiments, the learning of the patterns and relationships includes spatiotemporal propagation.

In some embodiments, determining the network response to stimuli includes the learning of the patterns and relationships of spatiotemporal propagation.

In some embodiments, determining a brain organoid behavior includes the learning of the patterns and relationships of spatiotemporal propagation.

Advantageously, based on the determined behavior, it may be, but not necessarily, easier to identify and predict a state of healthy or PD, than from the collective response/behavior of the network.

Also, a determined behavior is more prone to visualization through a visual simulation than the actual response of the network.

In some embodiments, the determined behavior comprises the response of the network to stimuli.

The term “simulated behavior” or “simulation” may be interchangeably used with the term “determined behavior” but may be more directed towards the whole process of recording data indicative of the network behavior/response, determining a behavior/response, and instructing to execute another stimuli session. The simulated behavior is the core process driven by the processor of the system/method that assesses the response of the network to one or more stimuli sessions.

In addition, the simulated behavior (or the determined behavior), may be visualized in a visual simulation using a simulation component such as a computer/screen/robot or the like.

The terms “determined behavior”, “determined response” and “simulated behavior” are interchangeable.

In some embodiments, simulating a behavior includes determining a behavior.

The terms “assessment of learning-behavior response associated with a psychiatric disorder (PD)”, “assessing PD”, and “assessment of the severity of a PD” may be interchangeably used.

According to some embodiments, assessment of a learning-behavior response associated with a PD-derived brain comprises assessing the severity of PD.

As used herein, the term “learning-behavior” refers to a learning process driven/mediated by the closed loop mode, involving stimuli and stimuli parameters, that are determined based on the organoid behavior determined in a former session(s) The learning process results from, and is reinforced by, repeatedly pairing a feedback stimulus with a preceding neural behavior and is herein computed as a change in the determined behavior/simulated behavior of a brain organoid (i.e., change in computational simulation of information indicative of the neuronal function/activity) in response to a positive or negative feedback treatment (i.e., feedback stimulus, elastic stimulus). The behavior response, including learning-behavior response is a computational functional analysis.

As used herein, the term “severity of PD” refers to assessment of a certain degree/level of PD. This is achieved by comparing the similarity of the determined brain organoids behavior or stem cell-derived 2D neuronal culture to predicted behaviors of plurality of PD-derived brain organoids comprising organoids having a range of PD severities, thereby augmenting the determined behavior to include a range of severity, wherein the obtaining of PD-derived brain organoid comprises organoids having a range of PD severities, and wherein the association of the labeled data with the PD-derived brain organoid comprises associating the labeled data with the range of PD severities; thereby augmenting the prediction behavior model to include a range of severities

As used herein, the term “psychiatric disorder (PD)” refers to a range of disorders that affect mental, emotional and/or behavioral aspects of a subject and may have a neurodevelopmental, neurodegenerative or neurological bases, in particular neurological and neurodevelopmental base, and encompass conditions associated with reduced cognitive function (i.e., cognitive functions associated with psychiatric disorder (PD)), characterized by, cognitive impairment/rigidity (e.g., adaptive learning, attention, memory), executive function (e.g. problem-solving, decision making, planning and organization), motivational aspects, social problems (e.g., social communication, social interaction) and/or repetitive and restricted patterns of behavior.

As used herein, the term “psychiatric disorder (PD)” may refer to the term “cognitive function associated psychiatric disorder (PD)”

In accordance, according to some embodiments, assessment of PD using the systems and methods of the invention encompass assessment of cognitive functions associated with psychiatric disorder (PD).

In some embodiments, the systems and methods provided herein include assessment of cognitive functions associated with psychiatric disorder (PD).

In some embodiments, the PD encompasses PD. In some embodiments, the PD encompasses cognitive functions associated with PD.

In some embodiments, the PD includes cognitive functions associated with PD.

In some embodiments, the PD includes conditions having neurodevelopmental, neurodegenerative, and/or neurological bases. each possibility is a separate embodiment.

In some embodiments, the neurodevelopmental, neurodegenerative, and/or neurological conditions include cognitive functions associated with psychiatric disorder (PD). Each possibility is a separate embodiment.

In some embodiments, the PD includes conditions having mental, emotional and/or behavioral aspects. each possibility is a separate embodiment.

In some embodiments, conditions having mental, emotional and/or behavioral aspects include conditions having cognitive functions associated with PD includes. each possibility is a separate embodiment.

In some embodiments, the PD or the cognitive functions associated with PD includes cognitive impairment/rigidity (e.g., adaptive learning, attention, memory), executive function (e.g. problem-solving, decision making, planning and organization), motivational aspects, social problems (e.g., social communication, social interaction) and/or repetitive and restricted patterns of behavior, or any combination thereof. Each possibility is a separate embodiment.

Without being bound to the theory non-limiting examples of PD include, Autism Spectrum Disorders (ASD), Bipolar disorder, Attention Deficit Hyperactivity Disorder (ADHD/ADD), Schizophrenia, Major Depression, Obsessive-Compulsive Disorders (OCD), and Epilepsy.

According to some embodiments, PD or condition having cognitive functions associated with PD comprises one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a different embodiment.

According to some embodiments, the PD or conditions having cognitive functions associated with PD are selected from one or more of Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a different embodiment.

As used herein, the term “Autism Spectrum Disorders” (“ASD”) refers to a range of neurodevelopmental disorders that mainly affect social and communication skills, but also include other symptoms related, for example, to learning and to repetitive behavior, and may further be associated with co-morbidities. The spectrum refers to the range of appearance of the disorder that can manifest very differently from person to person.

As used herein, the term “non-syndromic idiopathic ASD” (also known as idiopathic autism) refers to multifactorial, symptomatic-non genetic autism in which the etiology of the disorder is unknown and risk involves contributions of co-existing genetic and environmental factors.

According to some embodiments, the Autism Spectrum Disorders (ASD) comprises non-syndromic idiopathic ASD. According to some embodiments, non-genetic PD comprises non-syndromic idiopathic ASD.

The term “genetic psychiatric disorder” (“genetic PD”) refers to a psychiatric disorder in which a single mutation or a collection of mutations is known to lead to the development of pathology at a high probability (i.e., high-risk genetic markers are involved) with very minor or even completely without an involvement of environmental risk factors.

As used herein genetic psychiatric disorder (genetic PD) is distinguished from non-genetic psychiatric disorder (non-genetic PD).

As used herein, the term “non-genetic psychiatric disorder” (“non-genetic PD”) refers to a psychiatric disorder in which non-genetic factors fundamentally influence the risk and contribute to the etiology of the disorder along with low-risk genetic markers, therefore, especially in these disorders, a complex and multifactorial contributions of genes and environment co-exist, assumingly as early as prenatal development begins.

According to some embodiments, the psychiatric disorder (PD) comprises genetic psychiatric disorder (genetic PD). According to some embodiments, the psychiatric disorder (PD) comprises non-genetic psychiatric disorder (non-genetic PD).

As used herein, the term “organoid” refers to an in vitro, human pluripotent stem cells (hiPSC)-derived, grown, and to some level self-organized 3D tissue that resembles, at least in part of its structure, cell type composition, and/or functional qualities, an in vivo organ, for example, a brain. An organoid of the present invention may include a population of cells forming a brain organoid or a brain spheroid. The terms “brain organoid” and “brain spheroid” may be interchangeably used.

According to some embodiments, a brain organoid comprises a brain spheroid.

According to some embodiments, a brain organoid comprises 3D organoid and/or 2D cell culture derived therefrom. Each possibility is a separate embodiment.

According to some embodiments, a brain organoid comprises 3D organoid and/or 2D tissue derived therefrom. Each possibility is a separate embodiment.

According to some embodiments, a brain organoid comprises 3D organoid and/or 3D clamps, or spheroids derived therefrom. Each possibility is a separate embodiment.

According to some embodiments, the brain organoid comprises the tissue and/or cells thereof.

As used herein, the term “brain organoid” refers to a self-organized 3D at least partially structured tissue that is derived and generated from primary cells that are reprogramed and transformed into induced pluripotent stem cells (iPSC), and then differentiated into neural progenitor cells (NPC), and further differentiated into neurons. The brain organoids of the invention include cerebral or cortical organoids. The brain organoids of the invention include organoids derived from primary cells obtained from human individuals at all developmental stages, including from an embryo, a fetus, a newborn/neonate, a mature baby, a toddler, a child, a teen, or an adult.

In some embodiment, the systems and methods provided herein include assessment of neuronal networks.

In some embodiment, the neuronal networks may be 3D brain organoids or 2D cultures thereof generated by processing the 3D organoid after/during its formation (referring to “tissue and/or cells thereof”).

In other embodiments, the neuronal networks may be stem cell-derived 2D neuronal cultures.

In some embodiment, the systems and methods provided herein include assessment of 3D brain organoids, tissue and/or cells thereof in 2D culture, as well as stem cell-derived 2D neuronal cultures.

As used herein, the term “stem cell-derived 2D neuronal cultures” refers to 2D neuronal cultures generated/derived/prepared directly from iPSC or hESC without generating a brain organoid.

It is noted that the 2D cultures of “tissue and/or cells thereof” refers to 2D cultures derived from the 3D organoid after its formation, while “stem cell-derived 2D neuronal cultures” refers to neuronal culture derived directly from iPSC or hESC without generating a brain organoid.

In some embodiments, stem cell-derived 2D neuronal cultures include differentiated of iPSC to neurons.

In some embodiments, stem cell-derived 2D neuronal cultures include differentiated of hESC to neurons.

According to some embodiments, the system for assessment of a psychiatric disorder (PD) comprises a brain organoid and/or stem cell-derived 2D neuronal culture; Each possibility is a separate embodiment.

According to some embodiments, the method for assessment of a psychiatric disorder (PD) comprises obtaining a brain organoid and/or stem cell-derived 2D neuronal culture; Each possibility is a separate embodiment.

According to some embodiments, the method for training an AI algorithm for determining brain organoids behavior and/or determining stem cell-derived 2D neuronal culture behavior comprises obtaining a plurality of PD-derived brain organoid and a plurality of healthy brain organoids, and/or obtaining a plurality of PD-derived stem cell-derived 2D neuronal culture and a plurality of healthy stem cell-derived 2D neuronal culture; each possibility is a separate embodiment.

In some embodiments, the brain organoid is a cerebral or cortical organoid. Each possibility is a separate embodiment.

In some embodiments, the brain organoid is derived and generated from primary cells obtained from, for example, but not limited to, epithelial cells, fibroblasts, tissue-specific stem cells, nucleated blood cells, embryonic stem cells (hESCs), mesenchymal stem cells or hair keratinocytes. Each possibility is a separate embodiment.

In some embodiments, the primary cells obtained for generating a brain organoid include cells obtained from an embryo, a fetus, a newborn/neonate, a mature baby, a toddler, a child, a teen, and an adult, or any combination thereof. Each possibility is a separate embodiment.

The brain organoid may refer to organoids derived and generated from cells obtained from a subject with respect to whom it is undetermined/unknown if he is healthy with respect to PD, or if he suffers from at least one PD (‘undetermined cells’ ‘undetermined brain organoid’,). Or it may refer to organoids derived and generated from cells obtained from a subject who is healthy with respect to PD (‘healthy cell’, ‘healthy organoid’). Or it may refer to organoids derived and generated from cells obtained from a subject who has at least one PD (‘PD derived cells’, ‘PD derived organoid’).

In some embodiments, the brain organoid includes a healthy brain organoid. In some embodiments, the brain organoid includes a PD-derived brain organoid. In some embodiments, the brain organoid includes an undetermined brain organoid.

In some embodiments, the brain organoid is derived and generated from healthy cells. In some embodiments, the brain organoid is derived and generated from PD-derived cells. In some embodiments, the brain organoid is derived and generated from an undetermined cell.

In some the undetermined brain organoid includes an unknown severity of PD.

According to some embodiment, a brain organoid comprises one or more of an undetermined brain organoid, a PD-derived brain organoid, and a healthy brain organoid, or any combination thereof. Each possibility is a separate embodiment.

According to some embodiments, a brain organoid comprises cells obtained or derived from an embryo, a fetus, a neonate, a mature baby, a toddler, a child, a teen, or an adult. Each possibility is a separate embodiment.

According to some embodiments, cells obtained or derived from an embryo, a fetus, a neonate, a mature baby, a toddler, a child, a teen, or an adult comprise undetermined cells, PD-cells, or healthy cells. Each possibility is a separate embodiment.

The cells obtained for generating a brain organoid may be obtained by any of the herein described below methods for obtaining cells from a subject.

According to some embodiments, for the brain organoid is generated/derived from cells obtained from a subject using any one of Chorionic Villus Sampling (CVS), amniotic fluid test (Amniocentesis), in-vitro fertilization (IVF), post-mortem autopsy of an embryo or a fetus, a biopsy (e.g. puncture, scraping, swiping) of various tissues, cord blood collection or blood withdrawal, excretions or collection of body fluids, such as urine, stool, sputum, vomitus, or saliva, or obtained from hair samples. Each possibility is a separate embodiment.

According to some embodiments, a brain organoid comprises cells originally obtained from a subject with respect to whom it is undetermined/unknown whether he is a healthy subject or a suffering subject, or from a subject suffering from one or more psychiatric disorder (PD), or from a healthy subject.

As used herein the term, “PD-derived brain organoid” refers to a brain organoid generated from “PD-cells” which are cells obtained from a subject suffering from one or more psychiatric disorder (PD).

According to some embodiments, a PD-derived brain organoid comprises cells obtained from a subject suffering from one or more psychiatric disorders (PD).

As used herein, the term “healthy-derived brain organoid” refers to a brain organoid derived and generated from healthy cells which are cells obtained from a healthy subject.

According to some embodiments, a healthy-derived brain organoid comprises cells obtained from a subject not suffering from a psychiatric disorder (PD).

As used herein, the term “healthy” may refer to a subject, a brain organoid and/or a cell, and is used to distinguish a healthy state from a state of having a psychiatric disorder (PD) and a healthy state from a state of not knowing whether PD exists or not.

As used herein, the term “subject”, “patient” or “individual” may be used interchangeably and generally refer to a human, at any stage of human development including an embryo, a fetus, a neonate, a mature baby, a toddler, a child, a teen, or an adult. A subject may be a “healthy subject”, or a subject suffering from a psychiatric disorder (PD) (i.e., a “suffering subject”), or a subject with respect to whom it is undetermined/unknown whether he is a healthy subject or a suffering subject (i.e., an undetermined subject). For example, a subject may be an in vitro embryo or fetus that is a result of IVF.

According to some embodiments, the brain organoid is generated/derived from cells obtained from an embryo. According to some embodiments, the brain organoid is generated/derived from cells obtained from a fetus. According to some embodiments, the brain organoid is generated/derived from cells obtained from a neonate. According to some embodiments, the brain organoid is generated/derived from cells obtained from a mature baby. According to some embodiments, the brain organoid is generated/derived from cells obtained from a toddler. According to some embodiments, the brain organoid is generated/derived from cells obtained from a child. According to some embodiments, the brain organoid is generated/derived from cells obtained from a teen. According to some embodiments, the brain organoid is generated/derived from cells obtained from an adult.

In some embodiments, brain organoid is generated/derived from primary cells including for example, but not limited to, epithelial cells, fibroblasts, tissue-specific stem cells, nucleated blood cells, embryonic stem cells (hESCs), mesenchymal stem cells or hair keratinocytes.

In some embodiments, brain organoid is generated/derived from primary cells obtained from/by, for example, but not limited to, a blood withdrawal/blood test, or a biopsy (e.g. puncture, scraping, swiping) of various tissues.

In some embodiments, brain organoid is generated/derived from primary cells, obtained from, for example, but not limited to, excretions or collected body fluids, such as urine, stool, sputum, vomitus or saliva, or obtained from hair samples.

In some embodiments, brain organoid is generated/derived from primary cells, include epithelial cells, fibroblasts, tissue-specific stem cells, nucleated blood cells, embryonic stem cells (hESCs), mesenchymal stem cells, or hair keratinocytes. Each possibility is a separate embodiment.

In some embodiments, brain organoid is generated/derived from primary cells, obtained from excretions or collected body fluids, such as urine, stool, sputum, vomitus, or saliva, or obtained from hair samples, or from cells obtained by blood withdrawal/blood test, or a biopsy of a subject. Each possibility is a separate embodiment.

According to some embodiments, cells obtained to generate a brain organoid are obtained from an embryo, a fetus, or a neonate/newborn. According to some embodiments, cells obtained to generate a brain organoid are obtained from a mature baby. According to some embodiments, cells obtained to generate a brain organoid are obtained from a toddler (i.e., about 2-4 years). According to some embodiments, the cells obtained to generate a brain organoid are obtained from a child (i.e., about 5-12 years). According to some embodiments, the cells obtained to generate a brain organoid are obtained from a teen (i.e., about 13-19 years). According to some embodiments, the cells obtained to generate a brain organoid are obtained from an adult (i.e., older than >about 20 years). Each possibility is a separate embodiment.

As used herein, the term “adult” may also collectively refer to a toddler, a child, a teen, or an adult. According to some embodiments, adult comprises a toddler, a child, a teen, or an adult.

According to some embodiments, assessment of a psychiatric disorder (PD) comprises a brain organoid.

According to some embodiments, assessment of a psychiatric disorder (PD) comprises a brain organoid derived and generated from cells obtained from a subject with respect to whom it is undetermined whether he is a healthy subject or a suffering subject.

According to some embodiments, assessment of a psychiatric disorder (PD) comprises taking into consideration a predetermined learning-behavior response of a PD-derived brain organoid and/or a healthy brain organoid; each possibility is a separate embodiment.

According to some embodiments, assessment of a psychiatric disorder (PD) comprises a brain organoid generated from cells obtained from an embryo, a fetus or a neonate/newborn, a mature baby, a toddler, a child, a teen, or an adult. Each possibility is a separate embodiment.

As used herein, the term “mature baby” refers to the post-neonatal stage before the subject becomes a toddler (i.e., about 30 days-2 years).

As used herein, the term “prenatal” refers to the period and stages of human prenatal development that starts with fertilization and ends with birth. Prenatal development begins with embryonic development and continues in fetal development until birth. In accordance, the term prenatal may refer to an embryo or a fetus. In accordance, “prenatal cells” are derived from an embryo or a fetus.

As used herein, the term “embryo” refers to the initial stage of human development that begins just after fertilization of the female egg cell by the male sperm (i.e., gametes). An embryo may result from sexual intercourse, intrauterine insemination (IUI), or in vitro fertilization (IVF), including any process/type of assisted reproductive technology (ART) involved in fertility treatment, including but not limited to, for example, fertility medication, embryo transfer, intracytoplasmic sperm injection (ICSI), cryopreservation, assisted zona hatching (AZH), transvaginal ovum retrieval (OVR), and others.

As used herein, the term “fetus” refers to the stage of human development that begins from about the ninth week after fertilization and continues until birth.

The term “neonatal” refers to the period and stages that follow human pregnancy from the moment of birth of a newborn/neonate up to about 1 month after birth when the neonate becomes a mature baby. As used herein, a newborn/neonate also refers to a premature baby/preterm/premature infant. In accordance, “neonatal cells” are cells derived from a neonate. The terms, “newborn”, “premature infant” and “neonate” may be interchangeably used.

According to some embodiments, human prenatal cells are obtained by Chorionic Villus Sampling (CVS). According to some embodiments, human prenatal cells are obtained by amniotic fluid test (Amniocentesis). According to some embodiments, human prenatal cells are obtained by in-vitro fertilization (IVF). According to some embodiments, human prenatal cells are obtained by post-mortem autopsy of an embryo or a fetus.

According to some embodiments, human neonatal, mature baby, or adult cells are obtained by a biopsy (e.g. puncture, scraping, swiping) of various tissues. Each possibility is a separate embodiment. According to some embodiments, human neonatal cells are obtained from cord blood collection or blood withdrawal. According to some embodiments, human neonatal, mature baby, or adult cells are obtained from excretions or collected body fluids, such as urine, stool, sputum, vomitus, or saliva, or obtained from hair samples. Each possibility is a separate embodiment.

According to some embodiments, cells obtained for generating a brain organoid are obtained by biopsies of tissue stem cells such as, but not limited to, embryonic stem cells (hESCs), mesenchymal stem cells, nucleated blood stem cells, fibroblasts, epithelial cells, or keratinocytes. Each possibility is a separate embodiment.

In some embodiments, the brain organoid is a prenatal organoid. In some embodiments, the brain organoid is generated from cells obtained from an embryo or a fetus.

In some embodiments, the brain organoid is a neonatal organoid. In some embodiments, the brain organoid is generated from cells obtained from a neonate/newborn (less than 60 days old).

The generation of a brain organoid may rely on at least two different paths (i) brain organoid may be generated indirectly from iPSC by first differentiating them to NPC/neurons, or (ii) a brain organoid may be generated directly from iPSC.

The generation of a brain organoid relies on hiPSC ability to aggregate into embryonic bodies (EBs) and further self-organize into 3D structures that upon differentiation may contain multiple areas recapitulating/modulating an individual and specific region of the human brain or multiple different regions of the human brain, including but not limited to, for example, the cerebral region, the cortex, the forebrain, the midbrain, the retina, the hippocampus, the hypothalamus, the cerebellum, and other brain regions. A brain organoid includes a great diversity of differentiated cell types, including but not limited to, for example, neural progenitor cells (NPC), neurons, astrocytes, oligodendrocytes, and more, the differentiation of which can be unguided or unguided.

For example, “unguided differentiation” may result in a “cerebral brain organoid” that includes multiple areas of self-organize 3D structures that recapitulate and modulate the whole human brain including the cerebral region of the human brain and may include additional areas recapitulating other human brain regions, including a cortical area, the forebrain, and others. Alternatively, using a “guided differentiation” procedure, several types of “brain region-specific organoids” can be generated to recapitulate an individual region of interest of the human brain. A brain region-specific organoid includes uniform and reproducible tissue, for example, a “cortical neuroepithelium organoid” recapitulates only the cerebral cortex region of the human brain, a “forebrain organoid” and a “cortical spheroid organoid” also modulates only the cerebral cortex region of the human brain, a “midbrain organoid” modulates only the midbrain region of the human brain, and the like.

According to some embodiments, the brain organoid comprises multiple areas of self-organize 3D structures that recapitulate and modulate the whole human brain (i.e., cerebral brain organoid); according to some embodiments, the brain organoid comprises at least a cerebral area (i.e., cerebral brain organoid or brain region-specific organoids); according to some embodiments, the brain organoid comprises at least a cerebral area and a cortical area (i.e., cerebral brain organoid or brain region-specific organoids); according to some embodiments, the brain organoid consists of a cortical area only (“cortical brain organoid”); according to some embodiments, the brain organoid comprises at least a striatum area, at least an hippocampal area, at least a midbrain area, at least a cerebellum area, at least a spinal cord area, at least a hypo-thalamus area, at least a thalamus area, at least a basal ganglia area, at least a forebrain area, at least a midbrain area; according to some embodiments, the brain organoid comprises EB-like aggregates, cortical spheroids, cortical neuroepithelium or oligocortical spheroids. Each possibility is a separate embodiment.

In some embodiments, the prenatal and/or neonatal brain organoid includes one or more of midbrain organoid, hippocampal organoid, striatal organoid, neocortical organoid, cerebral organoid and/or cortical organoid or any combination thereof. Midbrain, hippocampal, striatal, neocortical. Each possibility is a separate embodiment.

According to some embodiments, the brain organoid comprises EB-like aggregates and/or spheroids. Each possibility is a separate embodiment.

According to some embodiments, the brain organoid comprises at least a striatum area, at least a hippocampal area, at least a midbrain area, at least a cerebellum area, at least a spinal cord area, at least a hypo-thalamus area, at least a thalamus area, at least a basal ganglia area, at least a forebrain area, at least a midbrain area, or any combination thereof. Each possibility is a separate embodiment.

According to some embodiments, brain organoids are generated using guided or unguided differentiation. Each possibility is a separate embodiment.

Methods have been developed for growing lab-grown neuronal and glial cell cultures from patients. Cultures may be formed in 2D, as a neuronal homogenous culture/tissue, some may be Hermogenes mixtures of neurons and other cell types such as micro-glia, astrocytes, blood cells. 3D neuronal cultures (organoids, spheroids, aggregated) are used in order to make a miniature structural representation of an organ. The 3D formation may be done by inducing guided or unguided developmental protocol, to control the maturation and differentiation process of the tissue, or the 3D formation may be a spontaneous assembly of structure. The 3D structure can contain, neuronal cells strictly, or a combination of different cells and cells origins.

The term “tissue and/or cells thereof” is related to the brain organoid and may refer to the tissue and/or cells used to generate the brain organoid in 2D/3D culture, prior to formation of the 3D brain organoid or it may refer to the tissue and/or cells used to generate the brain organoid in 2D/3D culture, after formation of the 3D brain organoid (i.e., the tissue and cells when forming a 3D structure). For example, tissue and/or cells thereof include cultured cells that take part, participate, in the process of forming a brain organoid, directly or indirectly, such as iPSC, NPC, or neurons, and remained in the culture without actually forming and being physically included in the formed brain organoid or spheroid. For example, a brain organoid may be generated indirectly from iPSC by first differentiating them to NPC/neurons, or a brain organoid may be generated directly from iPSC. Therefore, tissue and/or cells thereof include any “leftover” of cells in the culture.

It is noted that the 2D cultures of “tissue and/or cells thereof” refers to 2D cultures derived from the 3D organoid, namely, after its formation or during its formation, while “stem cell-derived 2D neuronal cultures” are neuronal culture derived directly from iPSC or hESC without generating brain organoid.

The term “tissue and/or cells thereof” may also refer to cells in 2D culture resulted from enzymatic, chemical, or mechanical processing of a 3D brain organoid. The processing may include enzymatic digestion, chemical degradation, or mechanical slicing of a brain organoid or spheroid into tissue slices in 2D culture or dissociated cells in 2D culture.

In some embodiments, the brain organoid comprises dissociated cells thereof in 2D culture. In some embodiments, the brain organoids comprise sliced tissue thereof in 2D culture.

In some embodiments, the brain organoid comprises processed tissue and/or cells thereof in 2D culture, wherein the processed tissue and/or cells thereof comprises sliced tissue and/or dissociated cells resulted from any one of enzymatic digestion, chemical degradation, or mechanical slicing of the brain organoid after 3D brain organoid formation.

According to some embodiments, the brain organoid comprises tissue and/or cells thereof; according to some embodiments, the tissue and/or cells thereof comprise population of cells prior to or after brain organoid formation; according to some embodiments, the tissue and/or cells thereof comprise 2D culture; according to some embodiments, the tissue and/or cells thereof comprise 3D structure/culture and/or brain organoid; according to some embodiments, the tissue and/or cells thereof comprise 2D culture resulted from enzymatic, chemical, or mechanical digestion or dissociation of a brain organoid. according to some embodiments, the tissue and/or cells thereof comprise 2D culture resulted mechanical slicing a brain organoid or spheroid into 2D slices. Each possibility is a separate embodiment. Each possibility is a separate embodiment.

According to some embodiments, a brain organoid comprises 3D organoid and/or 2D cell culture derived therefrom. Each possibility is a separate embodiment.

According to some embodiments, a brain organoid comprises 3D organoid and/or 2D tissue derived therefrom. Each possibility is a separate embodiment.

According to some embodiments, a brain organoid comprises 3D organoid and/or 3D clamps, or spheroids derived therefrom. Each possibility is a separate embodiment.

Non-limiting example of a 3D tissue/3D culture include Embryonic bodies, aggregates, brain organoids, and brain spheroids.

According to some embodiments, the brain organoid comprises 2D tissue and cells grown in culture; according to some embodiments, the brain organoid comprises a self-organized 3D structure grown in culture; according to some embodiments, the brain organoid comprises at least a cerebral and/or cortical tissue/area/region; according to some embodiments, the cerebral and/or cortical tissue comprises a defining 2D/3D structure, shape, and size and/or cell type composition, or combination thereof. Each possibility is a separate embodiment.

According to some embodiments, the method and system disclosed herein comprises 3D and/or 2D cultures comprising a brain organoid, and/or tissue and/or cells thereof derived and generated from cells obtained from a subject.

According to some embodiments, assessing PD by the method and system herein disclosed is performed in 2D culture using cells obtained from a subject and transformed to iPSC-derived neurons without generating a brain organoid.

As used herein, the term “obtaining” refers to a brain organoid, tissue, and/or cells thereof. The brain organoid or the tissue, and/or cells used to generate it, may be accepted, received, acquired, purchased from a third party and/or collected from a subject. The brain organoid, tissue, and/or cells obtained (hereinafter “the sample”) may be derived from a prenatal embryo or fetus, or neonatal newborn, or it may be derived from a mature baby, or it may be derived from an adult. The brain organoid, tissue, and/or cells obtained comprise cells originally collected from a subject.

In some embodiments, the obtaining of the brain organoid, tissue, and/or cells is performed by a different party than the party that utilizes the brain organoid according to the invention disclosed herein (i.e., by a third party); in some embodiments, the obtaining of the brain organoid, tissue, and/or cells may be performed by a third party that collects the sample from the subject and may store it or transfer it for storage with yet another different third party, until further use is performed with the sample according to the invention disclosed herein.

In some embodiments, the step of generating the brain organoid may be performed by a different party than the party who obtained the sample and/or transferred it to storage and/or utilizes the brain organoid according to herein disclosed invention; in some embodiments, the step of generating the brain organoid may be performed by the same party who obtained the sample and/or transferred it to storage.

The term “generating” refers to the in vitro procedure of producing in-culture a brain organoid, or spheroid, comprising tissue and cells derived from prenatal, neonatal, mature baby, or adult, undetermined, PD or healthy subjects. The procedure involves cell growth in culture and/or in a bioreactor, the transformation of cells, induction of pluripotency, cell expansion, cell aggregation, embryonic body formation, and differentiation.

According to some embodiments, the obtained brain organoid may be derived from any one or more a prenatal, neonatal, mature baby, or adult, undetermined, PD or healthy subjects.

According to some embodiments, obtaining a brain organoid comprises generating it by transforming the obtained human prenatal, neonatal, mature baby or adult, undetermined, PD or healthy cells to pluripotent stem cells (hiPSC). Each possibility is a different embodiment.

According to some embodiments, obtaining a brain organoid comprises generating it by transforming the obtained human prenatal, neonatal, mature baby, or adult, undetermined, PD or healthy cells to hiPSC-derived Neural Progenitor Cells (NPC). Each possibility is a different embodiment.

According to some embodiments, NPC are further differentiated.

According to an aspect, there is provided a system for assessment of a psychiatric disorder (PD), the system comprising:
(i) a brain organoid; (ii) a stimuli system capable of delivering stimuli/treatments to the brain organoid; (iii) a sensor coupled to a recorder capable of detecting and recording one or more signals indicative of neuronal function/activity of the brain organoid; (iv) a micro-controller unit (MCU) configured to receive, integrate and/or transmit data/information of the one or more signals; and (v) a computer/processor configured to:
   (a) send instructions to the stimuli system to provide one or more treatment/stimuli sessions, each session comprising a stimuli/treatment provided to the brain organoid; (b) obtain from the MCU data recorded in response to the one or more stimuli sessions, the data/information indicative of neuronal function/activity of the brain organoid; (c) determine a brain-organoids behavior based on the recorded data/information; and (d) apply an AI algorithm on the brain-organoids behavior to thereby classify the brain organoid based on a degree of similarity of the determined brain-organoids behavior to a predicted behavior of a PD-derived brain organoid and/or a healthy organoid.

In some embodiments, applying an AI algorithm on the brain-organoids behavior for classifying the brain organoid based on a degree of similarity of the data to a predicted behavior of a PD-derived brain organoid and/or a heathy organoid.

In some embodiments, the system comprises a brain organoid.

In some embodiments, the system comprises a stimuli system capable of delivering stimuli/treatments to the brain organoid.

In some embodiments, the system comprises a sensor coupled to a recorder capable of detecting and recording one or more signals indicative of neuronal function/activity of the brain organoid.

As used herein, the term “sensor” may refer to means of detecting electrophysiological signal or light signal, such as but not limited to electrodes or a microscope.

In some embodiments, the sensor comprises one or more multi-array electrodes (MAE) coupled to one or more recording head stage (RHS). In some embodiments, the sensor comprises an imaging device.

As used herein, the term “signals” may refer to electrophysiological measurements and imaging of light emitted from reporters, such as but not limited to genetic reporters for calcium influx, and the like, that may be used for detection and monitoring of neuronal function/activity.

In some embodiments, the one or more signal indicative of the neuronal function/activity of the brain organoid comprises an electrophysiological signal. In some embodiments, the one or more signal indicative of the neuronal function/activity of the brain organoid comprises a light signal.

In some embodiments, the system comprises a micro-controller unit (MCU) configured to receive, integrate and/or transmit data/information of the one or more signals.

As used herein, the term “data” is related to the term “signal” and may refer to any information indicative of neuronal function/activity of the brain organoid that is related to the signal detected or can be derived from it. In some embodiments, the information may include for example, but is not limited to: duration, intensity, frequency, amplitude, and/or spatial distribution/spatiotemporal propagation of the detected signal.

In some embodiments, information/data indicative of neuronal activity includes neuronal network response/activity in response to stimuli.

In some embodiments, information/data indicative of neuronal activity includes neuronal network response/activity in response to predetermined stimuli.

In some embodiments, information/data indicative of neuronal activity includes neuronal network response/activity in response to elastic stimuli; and wherein elastic stimuli is determined based on neuronal network response/activity to former stimuli. Each possibility is a separate embodiment.

In some embodiments, information/data indicative of neuronal includes spontaneous neuronal activity.

In some embodiments, data/information of the one or more signals includes information of electrophysiological recordings and/or reporter imaging. Each possibility is a separate embodiment.

In some embodiments, the data indicative of the neuronal function/activity comprises duration, intensity, frequency, amplitude, and/or spatial distribution/spatiotemporal propagation of the detected signal, or any combination thereof. Each possibility is a separate embodiment.

In some embodiments, the system comprises a computer/processor. In some embodiments, the computer/processor comprises an FPGA.

In some embodiments, the computer/processor is configured to send instructions to the stimuli system to provide one or more treatment/stimuli sessions.

In some embodiments, each stimuli session comprise a stimuli/treatment provided to the brain organoid.

In some embodiments, the computer/processor is configured to obtain from the MCU data recorded in response to the one or more stimuli sessions, the data/information indicative of neuronal function/activity of the brain organoid.

In some embodiments, the computer/processor is configured to determine a brain-organoid behavior based on the recorded data/information.

As used herein, the term “behavior” or “brain-organoid behavior” refers to neural function/activity in response to stimuli.

As used herein, the term “determined brain-organoid behavior” may refer to a behavior determined in response to a predetermined stimuli (open loop mode) or to a learning behavior determined in response to a stimuli determined based on the organoids behavior in one or more former sessions (i.e., elastic stimuli/closed loop mode.

In some embodiments, the determined brain-organoid behavior comprises a behavior determined in response to predetermined stimuli. (open loop mode)

In some embodiments, the determined brain-organoid behavior comprises a learning behavior determined in response to stimuli determined based on the organoids' behavior in one or more former sessions. (i.e., elastic stimuli/closed loop mode)

In some embodiments, the learning-behavior is determined in response to a change in the brain-organoids behavior between one or more former sessions and a later session. (closed loop mode)

In some embodiments, a stimuli determined based on the organoids behavior in one or more former sessions includes a positive or negative feedback treatment. (closed loop mode)

In some embodiments, the learning-behavior is determined in response to a positive or negative feedback treatment. (closed loop mode)

In some embodiments, the computer/processor is configured to apply an AI algorithm on the brain-organoids behavior to thereby classify the brain organoid based on a degree of similarity of the determined brain-organoid behavior to a predicted behavior of a PD-derived brain organoid and/or a heathy organoid. Each possibility is a separate embodiment.

The term “predicted behavior” refers to models of brain organoids behavior generated by the trained AI algorithm while learning a plurality of brain organoid responses to stimuli, provided thereto during one or more stimuli sessions. A predicted behavior may model a healthy-brain organoid behavior or PD-derived organoid behavior having a range of PD severities. (referring to the method of training).

In some embodiments, the behavior comprises a learning-behavior.

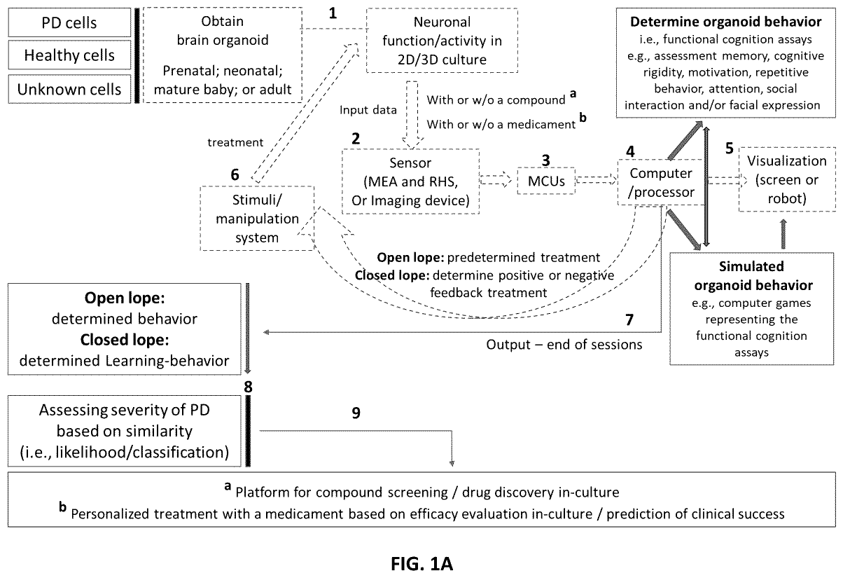

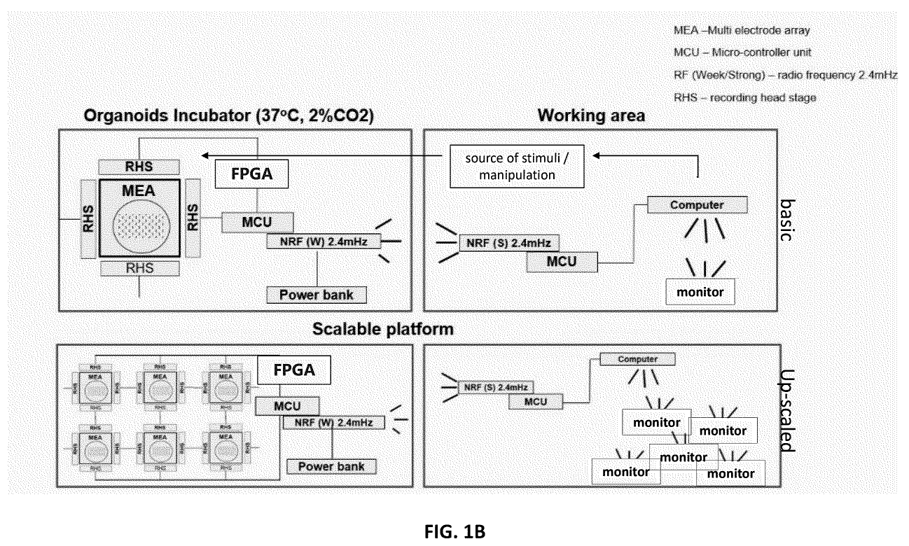

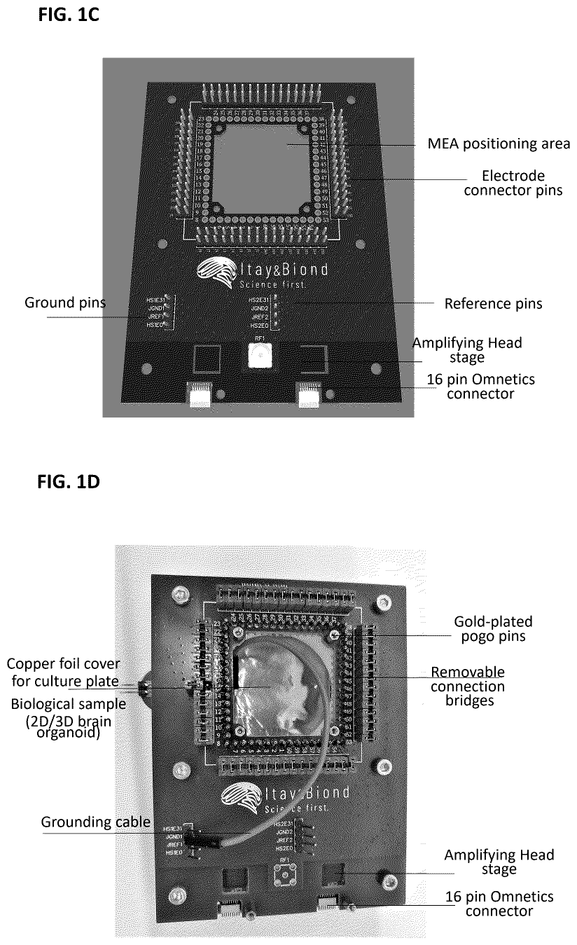

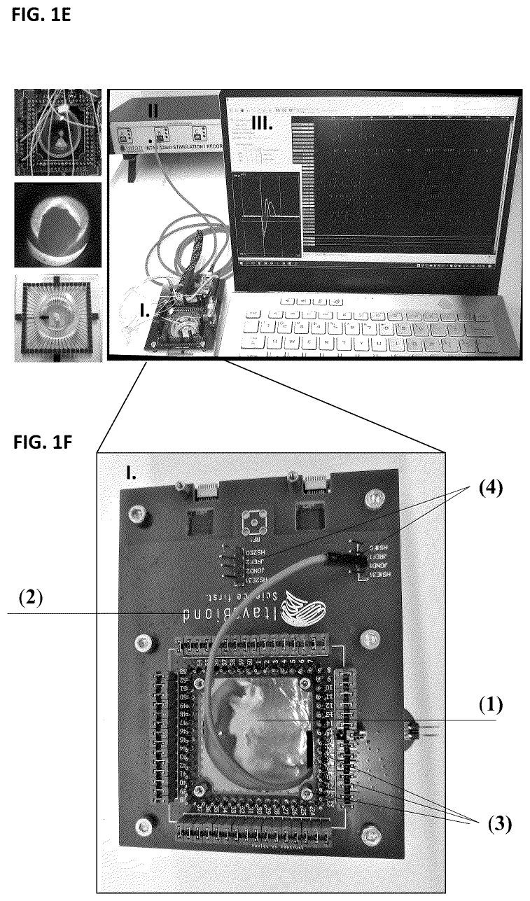

Reference is made to Example 1, FIG. 1A describing system components and illustrating the structure and function of the system.

According to some embodiments, the system being an open loop system, in which the stimulus provided to the brain organoid in the one or more sessions are predetermined.

According to some embodiments, the system includes an open loop system/mode, in which the treatment/stimulus, provided to the brain organoid in the one or more sessions are predetermined (predetermined stimuli).

According to some embodiments, the AI algorithm is trained on brain-organoids behaviors of a plurality of healthy and/or PD derived brain organoids in response to the predetermined treatment/stimulus.

In some embodiments, the training data is labeled according to one or more characteristics/parameters of the treatment/stimulus. (open loop)

As used herein, the term ““labeled” or “labeling” may relate to the AI-training process used in a closed and open system/modes and may refer to one or more characteristics/parameters of the treatment/stimulus, including but not limited to stimulus type, stimuli pattern/distribution and other treatment parameters such as intensity, duration, amplitude, frequency, and the like.

In the open system/mode, the labeling includes association of one or more characteristics/parameters of the predetermined stimuli with an organoid behavior that correspond to a healthy or PD-derived organoid (including a spectrum of PD-derived organoid representing different levels of PD severities).

In the closed system/mode, the labeling includes association of one or more characteristics/parameters of the elastic stimuli with an organoid behavior that correspond to a healthy or PD-derived organoid (including a spectrum of PD-derived organoid representing different levels of PD severities).

According to some embodiments, the AI algorithm is continuously reinforced, based on the determined brain-organoid behavior, to thereby improve the predicted behavior. (open loop)

As used herein, the term “open loop” refers to a mode of the system for assessing PD wherein the algorithm learns and classifies the behavior of the brain in response to predetermined stimuli.

The term “predetermined stimuli” is related to the term open loop, and refers to a predetermined, fixed treatment/stimuli that does not depend on the response of the brain organoid to previous stimuli. The stimuli and its parameters may or may not repeat themselves between sessions, but they are predetermined/fixed in that sense that when the parameters are set it is done without considering the response of the brain organoid to previous stimuli. Hence, predetermined stimuli stand in contrast to elastic stimuli.

The predetermined stimuli (open loop) or the elastic stimuli (closed loop) may have been labelled (possibly as part of the AI training process) according to one or more characteristics, including, for example, but not limited to the stimulus type, pattern and treatment parameters. The predetermined stimuli or the elastic stimuli may include a certain type of stimuli/treatment, or a combination thereof (e.g., electrophysiological pulse and light, or electrophysiological pulse and heat), which can be coordinated in parallel or in sequential pattern.

In some embodiments, the predetermined stimuli or the elastic stimuli have treatment/stimuli parameters including spatial pattern/distribution, intensity, duration, amplitude, frequency, concentration and/or temperature.

The types of the stimuli and the parameters of predetermined stimuli do not change based on the organoid behavior determined in response to the one or more former stimuli session provided, while those of elastic stimuli are adjusted according to the determined behavior or the response of the network. The predetermined stimuli may be simple or may have a more complex pattern, nevertheless the predetermined stimuli are preferably less complex than the “positive or negative feedback stimuli”.

Importantly, the predetermined stimuli or the elastic stimuli may have a corresponding brain organoid behavior that was predetermined during the AI training process; therefore, it may be associated with a predicted behavior or a range of predicted behaviors according to the model that was conceived/founded.

The terms “predetermined stimuli” and “fixed stimuli” may be interchangeably used.

Reference is now made to Example 1, FIG. 1A and Example 4—exemplifying an open loop system/mode.

According to some embodiments, the system being a closed loop system, in which the stimulus provided to the brain organoid is determined according to the determined brain-organoid behavior/response.

According to some embodiments, the system includes a closed loop system/mode, in which the treatment/stimulus provided to the brain organoid is determined according to the determined brain-organoid behavior (elastic stimuli).

According to some embodiments, the system includes a closed loop system, in which the treatment/stimulus provided to the brain organoid is a positive or negative feedback treatment/stimulus determined according to the determined brain-organoid behavior (elastic stimuli).

As used herein, the term “closed loop” refers to a mode of the system for assessing PD wherein the algorithm learns and classifies the learning-behavior of the brain in response to elastic stimuli.

As used herein, the term “elastic stimuli” is related to the term closed loop and refers to stimuli determined based on the organoid's behavior in one or more former sessions (e.g., positive or negative feedback stimuli).

In some embodiments, the determined brain-organoid behavior comprises a learning behavior.

In some embodiments, the determined brain-organoid behavior comprises a learning behavior determined in response to stimuli determined based on the organoid's behavior in one or more former sessions (i.e., elastic stimuli/closed loop).

In some embodiments, the learning-behavior is determined in response to a change in the brain-organoids behavior between one or more former sessions and a later session (closed loop).

In some embodiments, stimuli determined based on the organoids behavior in one or more former sessions includes a positive or negative feedback treatment (closed loop). Each possibility is a separate embodiment.

In some embodiments, the learning-behavior is determined in response to a positive or negative feedback treatment (closed loop). Each possibility is a separate embodiment.

According to some embodiments, the AI algorithm is trained on brain-organoids learning-behaviors of a plurality of healthy and/or PD derived brain organoids. (closed loop)

According to some embodiments, the AI algorithm is trained on brain-organoids learning-behaviors of a plurality of healthy and/or PD derived brain organoids in response to a positive or negative feedback treatment/stimulus.

In some embodiments, the training data is labeled according to one or more changes in parameters of the treatment/stimulus between session (closed loop).

In the close system/mode, the labeling includes association of a change in one or more characteristics/parameters with an organoid learning-behavior that correspond to a healthy or PD-derived organoid (including a spectrum of PD-derived organoid representing different levels of PD severities).

In some embodiments, the system performs at least two sessions.

In some embodiments, the stimuli provided in a latter session is determined based on the brain-organoids behavior determined in response to one or more former stimuli sessions (closed loop).

In some embodiments, the system performs at least two sessions, wherein the positive or negative feedback treatment/stimuli provided in a latter session is determined based on the brain-organoids behavior determined in response to one or more former stimuli sessions; thereby augmenting learning behavior response (closed loop).

In some embodiments, the stimuli provided in a latter session comprises a positive or negative feedback; and wherein a change in the brain-organoids behavior between a former and the latter sessions is indicative of a learning-behavior response of the brain organoid (closed loop).

In some embodiments, classifying the brain organoid is based on a degree of similarity of the learning-behavior response to a predicted learning-behavior response of a PD-derived brain organoid and/or of a healthy organoid (closed loop mode). Each possibility is a separate embodiment.

Reference is now made to Example 1, FIG. 1A and Example 5—exemplifying a closed loop system/mode.

According to some embodiments, the sensor comprises one or more multi-array electrode (MAE) coupled to one or more recording head stage (RHS).

According to some specific embodiments, the stimuli system and the multi-array electrode (MAE) are same or different. Each possibility is a separate embodiment.

In some embodiments, the MCU is connected to a wireless radio transmitter (RF) or a micro transmitter (MT) connecting it to at least one remote MCU. Each possibility is a separate embodiment.

In some related embodiments, the MCU is connected to a processor/computer or is an integral part thereof. In some embodiments, the processor comprises FPGA.

In further related embodiments, at least the MAE, RHS and a plate holder for culturing of the brain organoid are integrated in an all-in-one device.

In further related embodiments, at least the MAE, RHS and a plate holder for culturing of the brain organoid are fabricated in a single device.

In additional embodiments, the all-in-one device further comprises one or more of a source of stimuli, an MCU and/or a processor, or any combination thereof.

In some embodiments, the processor comprises FPGA.

Reference is now made to Examples 1-2, FIG. 1A-1E—illustrating and exemplifying the system components, in standard and scaled-up structure, and in an all-in-one device. In some embodiments, the obtained brain organoid is generated from one or more of prenatal cells, neonatal cells, cells of a mature baby, cells of a toddler, cells of a child, cells of a teen, and cells of an adult, or any combination thereof. Each possibility is a separate embodiment.

In some embodiments, the obtained brain organoid is generated from prenatal cells and/or neonatal cells.

According to some embodiments, the brain organoid is an undetermined brain organoid having unknown severity of PD.

According to some embodiments, the obtained brain organoid comprises 3D brain organoid in culture.

According to some related embodiments, the obtained brain organoid comprises tissue and/or cells thereof in 2D culture, and wherein the tissue and/or cells thereof include sliced tissue and/or dissociated cells resulted from any of enzymatic, chemical, and/or mechanical processing of a 3D brain organoid after it has formed, or any combination thereof. Each possibility is a separate embodiment.

In some embodiments, tissue and/or cells thereof include sliced tissue and/or dissociated cells resulted from any of enzymatic, chemical, and/or mechanical processing of a 3D brain organoid after it has formed, or any combination thereof. Each possibility is a separate embodiment.

According to some embodiments, the sensor comprises one or more multi-array electrode (MAE) coupled to one or more recording head stage (RHS)

In related embodiments, the one or more signal indicative of the neuronal function/activity of the brain organoid comprises an electrophysiological signal.

In related embodiments, the sensor comprises an imaging device.

According to some embodiments, the one or more signal indicative of the neuronal function/activity of the brain organoid comprises a light signal.

In some embodiments, the data/information indicative of neuronal function/activity of the brain organoid comprises electrophysiological recording and/or reporter imaging. Each possibility is a separate embodiment.

In some embodiments, the data/information indicative of neuronal function/activity of the brain organoid comprises information of long-term measurements.

In some embodiments, the stimuli/treatment comprises one or more of electrophysiological stimuli, optic/light stimulus, heat, a chemical agent/drug, or any combination thereof. Each possibility is a separate embodiment.

In some embodiments, the stimuli/treatment comprises electrophysiological stimuli.

In some embodiments, at least some of the processing is done with a field-programmable gate array (FPGA).

In some embodiments, the data indicative of the neuronal function/activity comprises duration, intensity, frequency, amplitude, and/or spatial distribution of the detected signal, or any combination thereof. Each possibility is a separate embodiment.

In some embodiments, the PD is selected from one or more of a neurological, neurodevelopmental and neurodegenerative condition, or any combination thereof. Each possibility is a separate embodiment.

In some embodiments, the neurological, neurodevelopmental and/or neurodegenerative condition is selected from one or more of: Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

In some embodiments, the PD comprises non-genetic PD.

In some embodiments, the PD comprises one or more diseases selected from Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

In some specific embodiments, the PD is Autistic Spectrum Disorder (ASD). In some further specific embodiments, the ASD is non-syndromic idiopathic ASD.

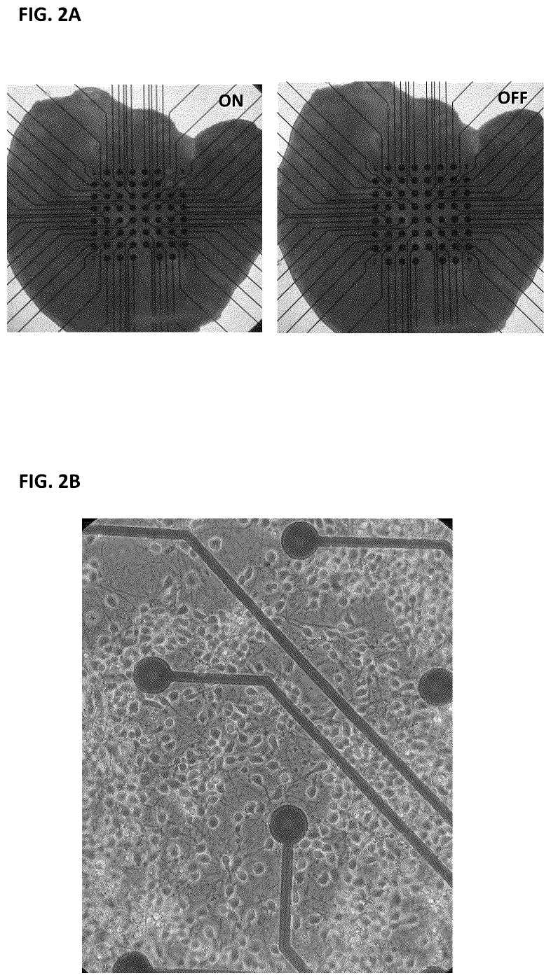

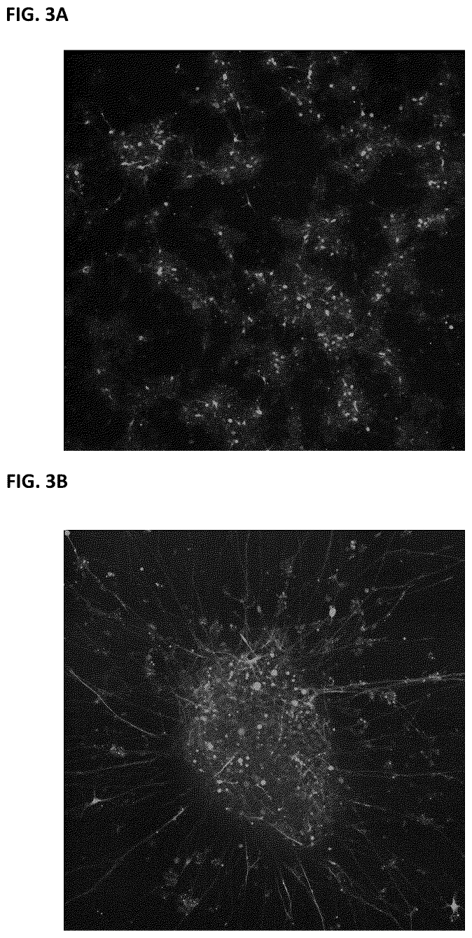

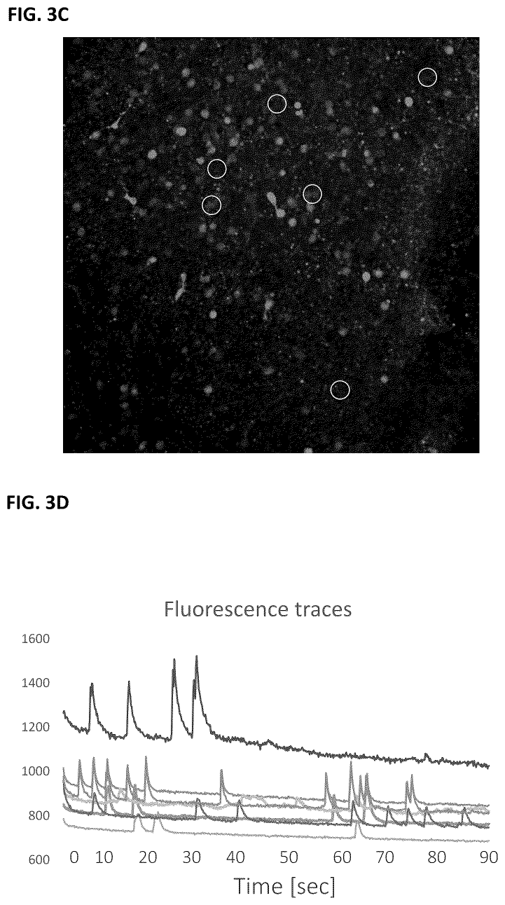

Reference is now made to Examples 2-3, FIGS. 2A-2B and 3A-3D exemplifying recordings of neural activity/function from ASD-derived organoids in 2D/3D culture using electrophysiological measurements and reporter imaging.

According to some embodiments, the system further comprises a visualization component presenting a visual simulation representative of a neural network functionality or of the determined organoid behavior.

According to some embodiments, the processor is further configured to visualize a simulation representative of a neural network functionality in response to stimuli or simulation representative of the determined organoid behavior. Each possibility is a separate embodiment.

According to some embodiments, the simulation includes, for example, but is not limited to a computer game.

In some embodiments, the simulation/visual simulation, or the computer game, or the processor, is configured to evaluate one or more of: memory, cognitive rigidity, motivation, repetitive behavior, attention, social interaction and/or facial expression, or any combination thereof. Each possibility is a separate embodiment.

According to some embodiments, the system further comprising a visualization component presenting a visual simulation representative of the determined organoid behavior.

In some embodiments, the visual simulation comprises a computer game.

In some embodiments, the computer game is configured to evaluate cognitive abilities selected from one or more of: memory, cognitive rigidity, motivation, repetitive behavior, attention, social interaction, processing speed, executive function, numerical abilities, and/or facial expression, or any combination thereof. Each possibility is a different embodiment.

In some embodiments, the visualization component is configured to present evaluation of cognitive abilities selected from one or more of: memory, cognitive rigidity, motivation, repetitive behavior, attention, social interaction, processing speed, executive function, numerical abilities, and/or facial expression, or any combination thereof. Each possibility is a different embodiment.

In some embodiments, cognitive assays/abilities are selected from one or more of: memory, cognitive rigidity, motivation, repetitive behavior, attention, social interaction, processing speed, executive function, numerical abilities, and/or facial expression, or any combination thereof. Each possibility is a different embodiment.

In some embodiments, the visualization component includes for example, but is not limited to one or more of a computer, a computer display, a mouse, a cursor, an artificial or prosthetic limb, a robot, or robotic device, or any combination thereof. Each possibility is a different embodiment.

In some embodiments, the further systems or methods comprise assessing the severity of PD based on the similarity.

As used herein, the term “similarity” refers to a comparison between a predicted behavior and a determined behavior.

In some embodiments, predicted behavior includes PD-like behavior and/or healthy-like behavior. Each possibility is a different embodiment.

In some embodiments the behavior comprises a learning-behavior (closed loop).

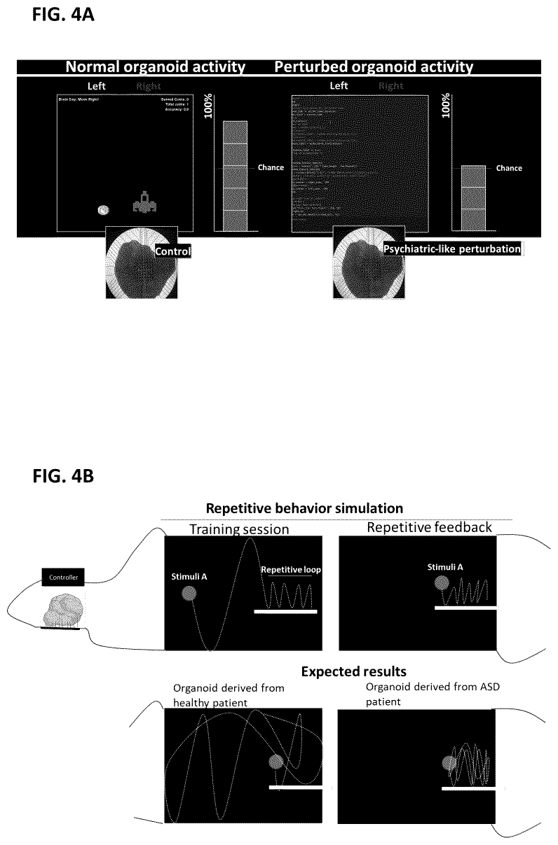

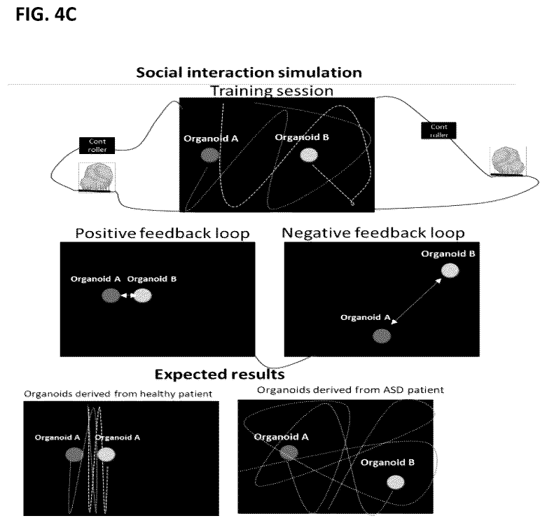

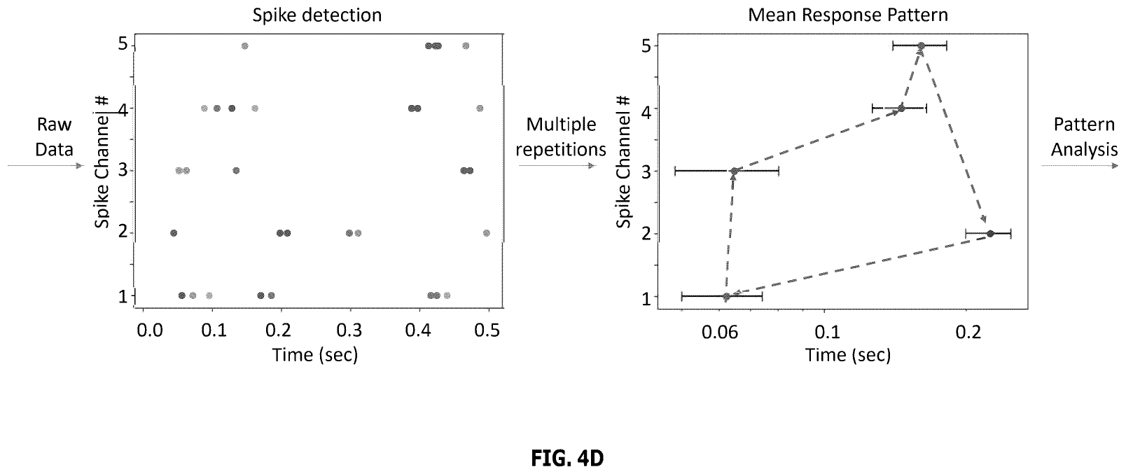

Reference is now made to Examples 4-5, FIGS. 4A-4C—exemplifying assessment of severity of PD using computer game simulations, in open loop and closed loop modes.

According to some embodiments, the processor is further configured to repeat steps a-c on the brain organoid after treatment thereof with a neurologic neurodevelopmental and/or neurodegenerative medicament, or any combination thereof. Each possibility is a separate embodiment.

According to some embodiments, the processor is further configured to repeat steps a-c on a brain organoid obtained from a same subject after neurologic neurodevelopmental and/or neurodegenerative treatment of said subject or any combination thereof.

In some embodiments, the neurological, neurodevelopmental and/or neurodegenerative treatment comprises a medicament. Each possibility is a separate embodiment.

In some embodiments, the neurological, neurodevelopmental and/or neurodegenerative treatment comprises a genetic treatment and/or electromagnetic treatment. Each possibility is a separate embodiment.

In some embodiments, the system further comprises determination of efficacy of the treatment. In some embodiments, the processor is further configured to determine efficacy of the treatment.

Reference is made to Example 6—describing personalized assessment of treatment efficacy by assessing PD severity of brain organoids.

According to another aspect, the disclosure provides a method for assessment of a psychiatric disorder (PD), the method comprising: (a) obtaining a brain organoid; (b) providing one or more treatment/stimuli sessions, each session comprising a stimuli provided to the brain organoid; (c) obtaining data recorded in response to the one or more treatment/stimuli sessions, the data/information indicative of neuronal function/activity of the brain organoid; (d) determining a brain-organoids behavior based on the recorded data; and (e) applying an AI algorithm on the brain-organoids behavior for classifying the brain organoid based on a degree of similarity of the determined brain-organoids behavior to a predicted behavior of a PD-derived brain organoid and/or a healthy organoid.

In some embodiments, applying an AI algorithm on the brain-organoids behavior for classifying the brain organoid based on a degree of similarity of the data to a predicted behavior of a PD-derived brain organoid and/or a healthy organoid. Each possibility is a separate embodiment.

According to some embodiments, the method includes an open loop method/approach, in which the treatment/stimulus and parameters thereof provided to the brain organoid in the one or more sessions are predetermined.

In some embodiments, the AI algorithm is trained on brain-organoids behaviors of a plurality of healthy and/or PD derived brain organoids in response to the predetermined treatment/stimulus, and wherein in a related embodiment, the training data is labeled according to one or more predetermined parameters of the treatment/stimulus (open loop). Each possibility is a separate embodiment.

In some embodiments, the AI algorithm is continuously reinforced, based on the determined brain-organoid behavior, to thereby improve the predicted behavior.

According to some embodiments, the method includes a closed loop method/approach, in which the treatment/stimulus provided to the brain organoid is determined according to the determined brain-organoid behavior (elastic stimuli; closed loop).

According to some embodiments, the method includes a closed loop method/approach, in which the treatment/stimulus provided to the brain organoid is a positive or negative feedback treatment/stimulus determined according to the determined brain-organoid behavior (elastic stimuli; closed loop).

In some embodiments, the AI algorithm is trained on brain-organoids behaviors of a plurality of healthy and/or PD derived brain organoids; and wherein in a related embodiment, the training data is labeled according to one or more parameters of the treatment/stimulus (closed loop).

In some embodiments, the AI algorithm is a reinforced learning algorithm trained on brain-organoids behaviors of a plurality of healthy and/or PD derived brain organoids in response to a positive or negative feedback treatment/stimulus, wherein the training data is labeled according to one or more parameters of the elastic treatment/stimulus (closed loop).

In some embodiments, the method comprises at least two sessions, wherein the treatment/stimuli provided in a latter session is determined based on the brain-organoids behavior determined in response to one or more former stimuli sessions.

In some embodiments, the method comprises at least two sessions, wherein the positive or negative feedback treatment/stimuli provided in a latter session is determined based on the brain-organoids behavior determined in response to one or more former stimuli sessions; thereby facilitating learning behavior response (positive or negative feedback).

In some embodiments, the stimuli provided in a latter session comprises a positive or negative feedback; and wherein in related embodiment, a change in the brain-organoids behavior between a former and the latter sessions is indicative of a learning behavior response of the brain organoid (closed loop).

In some embodiments, classifying the brain organoid is based on a degree of similarity of the learning-behavior response to a predicted learning-behavior response of a PD-derived brain organoid and/or of a heathy organoid (closed loop). Each possibility is a separate embodiment.

In some embodiments, the method further comprises generating a visual simulation representative of the determined organoid behavior.

In some embodiments, the visualization component is selected from a computer, a computer display, a mouse, a cursor, an artificial or prosthetic limb, a robot, or robotic device, or any combination thereof. Each possibility is a separate embodiment.

In some embodiments, the visual simulation comprises a computer game.

In some embodiments, the visual simulation comprises a computer game configured to evaluate one or more of: memory, cognitive rigidity, motivation, repetitive behavior, attention, social interaction, processing speed, executive function, numerical abilities, and/or facial expression, or any combination thereof. Each possibility is separate embodiment.

In some embodiments, the method further comprises assessing the severity of PD based on the similarity.

In some embodiments, the method further comprises repeating steps b-d on the brain organoid after treatment thereof with a neurological, neurodevelopmental and/or neurodegenerative medicament, or any combination thereof. Each possibility is a separate embodiment.

In some embodiments, the method further comprises repeating steps b-d on a brain organoid obtained from a same subject after neurological, neurodevelopmental and/or neurodegenerative treatment of said subject; and wherein the neurological, neurodevelopmental and/or neurodegenerative treatment comprises a medicament, a genetic or electromagnetic intervention, or any combination thereof. Each possibility is a separate embodiment.

In some embodiments, the method further comprises determining an efficacy of the treatment.

In some embodiments, the PD is selected from one or more of a neurological, neurodevelopmental and neurodegenerative condition, or any combination thereof. Each possibility is a separate embodiment.

In some embodiments, the neurological, neurodevelopmental and/or neurodegenerative condition is selected from one or more of: Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

In some embodiments, the PD comprises non-genetic PD.

In some embodiments, the PD comprises one or more diseases selected from Autism Spectrum Disorders (ASD), Bipolar disorder, Attention Deficit Hyperactivity Disorder (ADHD/ADD), Schizophrenia, Major Depression, Obsessive-Compulsive Disorders (OCD), Rett syndrome, Fragile X Syndrome, Intellectual Developmental Disorder, Down Syndrome, Williams Syndrome, Prader-Willi Syndrome, Angelman Syndrome, Smith-Magenis Syndrome, Epilepsy, Parkinson's disease, and Alzheimer's disease, or any combination thereof. Each possibility is a separate embodiment.

In some specific embodiments, the PD is Autistic Spectrum Disorder (ASD). In some further specific embodiments, the ASD is non-syndromic idiopathic ASD.

According to an aspect, there is provided a method for training an AI algorithm for determining organoids behavior, the method comprising:
(a) obtaining a plurality of PD-derived brain organoid and a plurality of healthy brain organoids; (b) providing one or more stimuli session(s), each session comprising stimuli provided to the brain organoid; (c) obtaining data recorded in response to the one or more treatment/stimuli session(s), the data is indicative of neuronal function/activity of the brain organoid; (d) labeling the data according to parameters of the one or more stimuli sessions and associating the labeled data with the PD-derived brain organoid and/or with the plurality of healthy brain organoid; (e) applying an AI algorithm on the data to learn patterns and relationships and to adjust parameters of a model for organoid behavior prediction; thereby training the algorithm for determining a brain-organoids behavior based on the data recorded in response to the one or more treatment/stimuli session(s).

In some embodiments, the AI algorithm is further trained to classify the organoids plurality of PD-derived brain organoids and/or healthy organoids based on the determined organoids' behavior as having ‘PD-derived behavior’ or a ‘heathy behavior’; thereby classifying the brain organoids based on a degree of similarity of their determined behavior to a predicted behavior of a PD-derived brain organoid and/or a heathy organoid.

In some embodiments, the obtaining of PD-derived brain organoid comprises organoids having a range of PD severities, and wherein the association of the labeled data with the PD-derived brain organoid comprises associating the labeled data with the range of PD severities; thereby augmenting the prediction behavior model to include a range of severities.

In some embodiments, the data indicative of neuronal function/activity of the brain organoid is divided to a ‘training dataset’ and ‘validation set’, and wherein the ‘validation set’ comprises unlabeled data used to improve model performance.

In some embodiments, wherein the AI algorithm includes one or more of supervised learning, unsupervised learning, semi-supervised learning, reinforced learning, self-supervised learning, transfer learning, meta-learning, evolutionary algorithms, or any combination thereof. Each possibility is a separate embodiment.

In some embodiments, the AI algorithm is a supervised machine learning algorithm. In some embodiments, the AI algorithm is a supervised machine learning algorithm capable of regression and/or classification. Each possibility is a separate embodiment.

In some embodiments, the AI algorithm is a supervised machine learning algorithm capable of regression and/or classification and includes one or more of Support-vector machines, Linear regression, Logistic regression, Random Forest, Naive Bayes, Linear discriminant analysis, Decision trees, K-nearest neighbor algorithm, Deep Neural networks, Neural networks (Multilayer perceptron), Gradient Boosting Algorithms, Linear Discriminant Analysis, Ridge Regression and Lasso Regression, Elastic Net, Bayesian Regression, Multiclass Classification Algorithms, and Similarity learning, or any combination thereof. Each possibility is a separate embodiment.

In some embodiments, the AI algorithm includes a supervised machine learning algorithm capable of regression and/or classification, including for example, but not limited to: Analytical learning, Artificial neural network, Backpropagation, Boosting (meta-algorithm), Bayesian statistics, Case-based reasoning, Decision tree learning, Inductive logic programming, Gaussian process regression, Genetic, programming, Group method of data handling, Kernel estimators, Learning automata, Learning classifier systems, Learning vector quantization, Minimum message length (decision trees, decision graphs, etc.), Multilinear subspace learning, Naive Bayes classifier, Maximum entropy classifier, Conditional random field, Nearest neighbor algorithm, Probably approximately correct learning (PAC) learning, Ripple down rules, a knowledge acquisition methodology, Symbolic machine learning algorithms, Subsymbolic machine learning algorithms, Support vector machines, Minimum complexity machines (MCM), Random forests, Ensembles of classifiers, Ordinal classification, Data pre-processing, Handling imbalanced datasets, Statistical relational learning, Proaftn, and a multicriteria classification algorithm, or any combination thereof. Each possibility is a separate embodiment.

In some embodiments, AI algorithm includes a machine learning classification algorithm, including for example, but not limited to Support vector machines, K-Nearest Neighbours, Decision trees, Artificial neural networks, Logistic regression, I Bayes, Linear Discriminant Analysis (LDA), and Quadratic Discriminant Analysis (QDA), or any combination thereof. Each possibility is a separate embodiment.

In some embodiments, the AI algorithm is a reinforced learning algorithm, including for example, but not limited to Q-Learning, Deep Q-Network (DQN), Policy Gradients, Actor-Critic, Model-Based RL, Deep Deterministic Policy Gradient (DDPG), Twin Delayed DDPG (TD3), Monte Carlo Methods, SARSA (State-Action-Reward-State-Action), Multi-Agent RL, Hindsight Experience Replay (HER), Exploration Strategies, Off-Policy Algorithms, and Imitation Learning, or any combination thereof. Each possibility is a separate embodiment.

In some embodiments, the AI algorithm is a classification algorithm. In some embodiments, the AI algorithm is a reinforced learning algorithm.

In some embodiments, the method comprising open loop training mode, wherein the treatments/stimuli provided to the brain organoid in the one or more sessions are predetermined.

In some embodiments, the method comprising closed loop training mode, wherein the treatments/stimuli provided to the brain organoid in the one or more sessions is determined according to the determined brain-organoid behavior.

In some embodiments, the stimuli provided to the brain organoid comprises one or more of an electrophysiological stimulus, a heat stimulus, a light stimulus, or a drug, or any combination thereof. Each possibility is a separate embodiment.

In some embodiments the stimuli provided to the brain organoid comprises an electrophysiological stimulus.

In some embodiments at least the plurality of healthy brain organoids is generated from prenatal or neonatal cells (cells obtained from an embryo, a fetus, or a newborn (i.e., up to about 60 days after birth), or any combination thereof. Each possibility is a separate embodiment.

In some embodiments, the plurality of PD-derived brain organoids is generated from prenatal or neonatal cells (cells obtained from an embryo, a fetus, or a newborn (i.e., up to about 60 days after birth), or any combination thereof. Each possibility is a separate embodiment.

In some embodiments, the training of the AI algorithm for determining organoids behavior includes determining prenatal and/or neonatal organoids behavior.

In some embodiments, the learning of the patterns and relationships includes one or more of spatiotemporal propagation including duration or distribution of the signal, intensity, frequency, and amplitude, or any combination thereof.

In some embodiments, the learning of the patterns and relationships includes spatiotemporal propagation

In some embodiments, determining the network response to stimuli includes the learning of the patterns and relationships of spatiotemporal propagation.

In some embodiments, determining a brain organoid behavior includes the learning of the patterns and relationships of spatiotemporal propagation.

According to some embodiments, the invention provides a method for training an AI algorithm for determining one or mor signal(s) and/or attribute(s), the method comprising:
(a) obtaining a plurality of PD-derived brain organoid and a plurality of healthy brain organoid; (b) obtaining data recorded of one or mor signal(s) and/or attribute(s) labeling the data according to parameters of the one or mor signal(s) and/or attribute(s) and associating the labeled data with the PD-derived brain organoid and/or with the plurality of healthy brain organoid; (c) applying an AI algorithm on the data to learn patterns and relationships and to adjust parameters of a model; thereby training the algorithm for determining one or mor signal(s) and/or attribute(s). each possibility is a separate embodiment.

Referring to the principal components of the herein provided non-limiting illustration of which is presented in FIG. 1A-1B—The neural recording and stimulation setup described involves the following components:
Multi-Electrode Arrays (MEA): these arrays are used for recording and stimulating neural activity.
Headstage: This element has two non-limiting functions: 1. Converting analog neural signals to digital format for processing and vice versa. 2. Signal Processing function: including filtering, amplification, and optional basic Digital Signal Processing (DSP) capabilities for preprocessing the recorded neural data.
FPGA (Field-Programmable Gate Array) or Equivalent Element: This component is responsible for high-speed parallel signal processing tasks, such as spike detection and classification, as well as the synchronization of stimulation triggers.
Microcontroller Unit (MCU): The MCU executes various programs and tests. It is connected to the FPGA and can execute higher-level programming languages like Python and C. When dealing with a limited number of electrodes, the FPGA functions can be implemented in the MCU, and the FPGA can be removed, as extreme parallelism is not required for simple experiments.
Data Output and Control: The programs executed on the MCU can be used to control the setup and broadcast the output data over RF (Radio Frequency), wired connections, or store it on local disk storage.
   In summary, this neural recording and stimulation setup comprises MEA arrays for neural signal acquisition, Headstage (signal processing and amplification), an FPGA (Field-Programmable Gate Array) or Equivalent Element for high-speed parallel processing, an MCU for program execution and control, and various options for data output and storage, Stimuli source and optionally computer/processor for additional data processing and/or analysis, and visualization system. Depending on the complexity of the experiment and the number of electrodes used, the FPGA may be optional, with the MCU capable of handling simpler tasks.

According to an aspect of the disclosure, there is provided a system for assessment of a learning-behavior response associated with a psychiatric disorder (PD), the system comprising, (i) a brain organoid; (ii) a stimuli/manipulation system capable of delivering treatment to the PD-derived brain organoid; (iii) a sensor coupled to a recorder capable of detecting and recording one or more signals of the brain organoid; (iv) at least one micro-controller unit (MCU) configured to receive, integrate and/or transmit information/data indicative of neuronal function/activity derived from the one or more signals; (v) a computer/processor connected to and/or running a simulator and configured to: (a) obtain input data comprising the information indicative of neuronal function/activity from the brain organoid; (b) apply an algorithm to generate a simulation of the brain-organoids behavior (i.e., the neuronal function/activity of step b); (c) determine a positive or negative feedback treatment based on the simulated behavior; (d) send instructions to stimuli/manipulation system to execute the determined positive or negative feedback treatment; (e) obtain post-treatment input data from the brain organoid comprising information indicative of neuronal function/activity in response to the execution of the determined positive or negative feedback treatment; (f) apply the algorithm to simulate a post-treatment behavior of the brain organoid data; (g) repeating steps (c) to (f) X times wherein X is an integer between 1 and 10000; and (h) compute an output score indicative of the learning-behavior response based on a change in the behavior of the PD-derived brain organoid; and (i) assesses the severity of PD based on similarity to a predetermined learning-behavior response of a PD-derived brain organoid and/or predetermined learning-behavior response of a healthy brain organoid.

In some embodiments, X is an integer between 1-10000, 1-1000, or 1-100. Each possibility is a separate embodiment.

According to some embodiments, the computer/processor comprises a simulator.

According to some embodiments, the system comprises at least two components that operate in parallel or partially in parallel, at least three components that operate in parallel or partially in parallel, at least four components that operate in parallel or partially in parallel.

According to some embodiments, the method comprises at least two steps that performs in parallel or partially in parallel, at least three steps that performs in parallel or partially in parallel, at least four steps that performs in parallel or partially in parallel.

For example, according to some embodiments, the obtaining of post-treatment input data of step (e) continues through the simulation of step (f) and may be at least partially performed in parallel.

Advantageously, the herein-disclosed system and method provide an assessment of a learning-behavior response associated with PD utilizing a brain organoid.

Accordingly, further advantageous, the evaluation of the learning-behavior response associated with the a brain organoid may be indicative of the severity of PD and/or may be used to evaluate the clinical success of treatment with a psychiatric/neurologic drug.

According to one aspect, the present disclosure provides loop system for assessment of a learning-behavior response associated with a psychiatric disorder (PD).

According to some embodiments, the system comprising:
(i) a brain organoid;
(ii) a stimuli/manipulation system capable of delivering treatment to the brain organoid;
(iii) a sensor coupled to a recorder capable of detecting and archiving/recording one or more signals of the brain organoid;
(iv) at least one micro-controller unit (MCU) configured to receive, integrate and/or transmit information/data indicative of neuronal function/activity derived from the one or more signals;
(v) a computer/processor connected and/or running to a simulator.

According to some embodiments, the computer/processor comprises a simulator.

According to some embodiments, the stimuli/manipulation system is capable of delivering treatment to a brain organoid; according to some embodiments, the stimuli/manipulation system is capable of delivering treatment to a brain organoid in culture.

As used herein, the terms “treatment” or “stimuli” may refer to one or more of electrical pulse (electrophysiological stimuli), optic/light stimulus, heat, a chemical agent/drug, or any combination thereof. The treatment/stimuli may be delivered to a brain organoid in culture by the stimuli/manipulation system. The terms “treatment” or “stimuli” may be interchangeably used.

According to some embodiments, the stimuli/manipulation system is capable of delivering to a brain organoid, one or more treatments comprising an electrical pulse, optic/light stimulus, heat, or a chemical agent/drug, or any combination thereof; according to some embodiments, the stimuli/manipulation system is capable of delivering to a brain organoid a treatment comprising an electrical pulse (electrophysiological stimuli); according to some embodiments, the stimuli/manipulation system is capable of delivering to a brain organoid a treatment comprising an optic/light stimulus or heat; according to some embodiments, the stimuli/manipulation system is capable of delivering to a brain organoid a treatment comprising a chemical agent/drug/medicament. Each possibility is a separate embodiment.

According to some embodiments, the stimuli/manipulation system comprises an electrophysiology system. According to some embodiments, the stimuli/manipulation system capable of delivering electric pulse, and the sensor comprising a multi-array electrode (MAE) are same or different.

According to some embodiments, the sensor coupled to a recorder capable of detecting and archiving/recording the one or more signals comprises a multi-array electrode (MAE) coupled to one or more recording head stages (RHS); according to some embodiments, the sensor coupled to a recorder capable of detecting and archiving/recording one or more signals comprises an imaging device coupled to a camera.

According to some embodiments, at least one micro-controller unit (MCU) is configured to receive, integrate and/or transmit information/data indicative of neuronal function/activity derived from one or more signals. Each possibility is a separate embodiment.

According to some embodiments, at least one micro-controller unit (MCU) is an integral part of the computer/processor.

According to some embodiments, the information indicative of the neuronal function/activity is transferred from the sensor coupled to a recorder to at least one MCU; according to some embodiments, the MCU is connected to a wireless radio transmitter (RF) or a micro transmitter (MT) connecting it to at least one remote MCU; according to some embodiments, wherein the MCU is connected to a processor/computer. Each possibility is a separate embodiment.

According to some embodiments, at least one signal detected and archived is an electric signal or an optic/light signal; according to some embodiments, the optic/light signal detected is omitted from a genetic reporter; according to some embodiments, the genetic reporter capable of omitting optic/light signal comprises Ca⁺² imaging reporter or Redox imaging reporter; according to some embodiments, at least one electric signal or optic/light signal detected and archived comprises information indicative of the neuronal function/activity of a PD-derived brain organoid. Each possibility is a separate embodiment.

According to some embodiments, the at least one signal detected and archived comprises information of long-term measurements.

According to some embodiments, the system comprises components for maintaining a brain organoid, tissue and/or cell thereof in culture. Non-limiting examples components for maintaining a brain organoid, tissue and/or cell thereof in culture include an incubator, a temperature controller, a CO2 and oxygen controller.

As used herein, the term “long-term measurement” refers to an electrophysiological measurement performed by the system after a stimuli was delivered to the brain organoid, and persists for longer than 5 min. Non limiting example of a stimuli for long term measurements includes a drug or heat.

In some embodiments, a stimuli/treatment for long-term measurement one or more of electrophysiological stimuli, a drug, heat, light, or any combination thereof. Each possibility is a separate embodiment.

According to some embodiments, a long-term measurement persists for at least 5 min, at least 10 min, at least, 30 min, at least 60 min, at least 2 hours, at least 12 hours, at least 24 hours, at least several days, at least a week, at least several weeks, at least 1 month, at least 2 months, at least 6 months, or at least a year. Each possibility is a separate embodiment.

According to some embodiments, an electrophysiological measurement comprises at least a single ‘short term’ measurement of up to 5 min, at least two ‘short term’ measurements of up to 5 min each, at least three ‘short term’ measurements of up to 5 min each, at least four ‘short term’ measurements of up to 5 min each, at least five ‘short term’ measurements of up to 5 min each, at least ten ‘short term’ measurements of up to 5 min each. Each possibility is a separate embodiment.

According to some embodiments, an electrophysiological measurement comprises at least a single short-term measurement followed by a long-term measurement.

According to some embodiments, an electrophysiological measurement comprises at least a single long short term measurement, at least two consecutive cycles of long short term measurement, at least three consecutive cycles of long short term measurement, at least four consecutive cycles of long short term measurement, at least five consecutive cycles of long short term measurement, at least ten consecutive cycles of long short term measurement. Each possibility is a separate embodiment.

According to some embodiments, the system comprises a computer/processor connected to and/or running a simulator and configured to:
a. obtain input data comprising the information indicative of neuronal function/activity from the brain organoid;
b. apply an algorithm to generate a simulation of the brain-organoids behavior/neuronal function/activity;
c. determine a positive or negative feedback treatment based on the simulated behavior;
d. send instructions to stimuli/manipulation system to execute the determined positive or negative feedback treatment;
e. obtain post-treatment input data from the brain organoid comprising information indicative of neuronal function/activity in response to the execution of the determined positive or negative feedback treatment;
f. apply the algorithm to simulate a post-treatment behavior of the brain organoid data;
g. repeating steps (c) to (f) X times; wherein X is an integer between 1 and 100;
h. compute an output score indicative of the learning-behavior response based on a change in the behavior of the PD-derived brain organoid; and
i. assess the severity of PD based on similarity to a predetermined learning-behavior response of a PD-derived brain organoid and/or predetermined learning-behavior response of a healthy brain organoid. Each possibility is a separate embodiment.

According to some embodiments, the computer/processor connected to and/or running a simulator is configured to obtain input data comprising the information indicative of neuronal function/activity from the brain organoid. Each possibility is a separate embodiment.

As used herein, the term “input data” refers to information recorded which is indicative of neuronal function/activity before treatment (i.e., behavior of a brain organoid). As used herein, the term “post-treatment input data” refers to (i) information indicative of neuronal function/activity in response to execution of a determined positive or negative feedback treatment (i.e., post-treatment behavior of a brain organoid), and/or refers to (ii) information indicative of parameters of the determined and/or executed positive or negative feedback treatment.

As used herein, the term “post-treatment behavior” refers to information indicative of neuronal function/activity in response to execution of a determined positive or negative feedback treatment.

In some embodiments, the input data is obtained prior to any treatment delivered to the brain organoid (i.e., basal state, resting state). In some embodiments, the post-treatment input data is obtained after the delivery of treatment/in response to execution of a determined positive or negative feedback treatment to the brain organoid.

In some embodiments, the input data comprises information indicative of the neuronal function/activity of the brain organoids in culture before treatment; in some embodiments, the post-treatment input data comprises information indicative of the neuronal function/activity of a brain organoid in culture after treatment; in some embodiments, the post-treatment input data comprises information indicative of the neuronal function/activity of a brain organoid in culture in response to execution of a determined positive or negative feedback treatment to the brain organoid; in some embodiments, post-treatment input data comprises the post-treatment behavior of a brain organoid.

In some embodiments, the input data comprises additional information, non limiting examples being, biological data and/or physical data and/or molecular data and/or biochemical data and/or metabolic data and/or information on origin of the subject.

In some embodiments, the post-treatment input data comprises information indicative of parameters of the determined positive or negative feedback treatment delivered by the stimuli/manipulation system to the brain organoids in culture.

As used herein, the term “positive or negative feedback treatment” may refer to one or more of an electrical pulse, optic/light stimulus, heat, a chemical agent/drug, or any combination thereof.

According to some embodiments, the determined positive or negative feedback treatment delivered to the brain organoid in culture by the stimuli/manipulation system comprises electrical pulse, optic/light stimulus, heat, or a chemical agent/drug, or any combination thereof. Each possibility is a separate embodiment.

According to some embodiments, the MAE comprises electric signal generated in response to light. (as in photo-stimulated MAE with the electrodes having a semiconducting component). According to some embodiments, the computer/processer is connected to light source which illuminate specific electrodes on the MAE.

The term “parameters” may refer to concentration, temperature, duration, intensity, spatial distribution/spatiotemporal propagation, frequency and/or amplitude of the stimuli/treatment (whether predetermined or determined positive or negative feedback.

The term “information indicative of parameters” refers to the values of the parameters of the determined positive or negative feedback treatment.

In some embodiments, information indicative of the neuronal function/activity comprises spatiotemporal propagation, duration, intensity, frequency and/or amplitude of the detected signal; in some embodiments, the information indicative of the neuronal function/activity further comprises spatial information.

According to some embodiments, the treatment delivered by the stimuli/manipulation system to the brain organoid is one or more of an electrical pulse, optic/light stimulus, heat, a chemical agent/drug, or any combination thereof.

In some embodiments, the information indicative of parameters of the determined positive or negative feedback treatment delivered by the stimuli/manipulation system to the brain organoids in culture comprises information about spatiotemporal propagation, concentration, temperature, duration, intensity, frequency and/or amplitude of the stimuli.

According to some embodiments, the computer/processor comprises a simulator.

According to some embodiments, the computer/processor connected to and/or running a simulator is configured to apply an algorithm to generate a simulation of the brain-organoids behavior.

In some embodiments, the algorithm that generates a simulation of the brain-organoids behavior is a reinforcement learning algorithm According to some embodiments, the computer/processor connected to and/or running a simulator is configured to determine a positive or negative feedback treatment based on the simulated behavior.

According to some embodiments, the computer/processor connected to and/or running a simulator is configured to send instructions to stimuli/manipulation system to execute the determined positive or negative feedback treatment.

According to some embodiments, the computer/processor connected to and/or running a simulator is configured to obtain post-treatment input data from the brain organoid comprising information/data indicative of neuronal function/activity in response to the execution of the determined positive or negative feedback treatment.

According to some embodiments, the computer/processor connected to and/or running a simulator is configured to apply the algorithm to simulate a post-treatment behavior of the brain organoid data.

Non-limiting examples of a simulator/visualization component include a computer display, a monitor, a mouse, a cursor, an artificial or prosthetic limb, a robot, or robotic device

As used herein, the term “psychiatric-related computer games” refers to computer games used in evaluation of social interaction, repetitive behavior, cognitive rigidity, and/or face recognition and utilized for the simulation of brain organoid behavior.

Non-limiting examples of psychiatric-related computer games used for simulating PD-derived brain organoid behavior include:
(i) Social interaction games/simulations-examining the capacity of a brain organoid to learn social interaction abilities. For example-gaining positive feedback when acting together or as a function of success and negative for failure or “operating alone”.
(ii) Face recognition games/simulations—may include for example positive feedback for a “happy smile” or simple objects, non-limiting examples being numbers, letters or shapes.
(iii) Repetitive behavior games/simulations-examining the repetitive behavior of a brain organoid and the capacity to change it.
(iv) Cognitive rigidity games/simulations-examining the capacity of a brain organoid to learn a task and reflect from it to a different task.

According to some embodiments, the output score indicative of the learning-behavior response based on a change in the behavior of the brain organoid comprises a combination of scores achieved in different games and/or time points.

According to some embodiments, the simulated behavior comprises information/data indicative of the neuronal function/activity

According to some embodiments, the simulator comprises one or more of a computer, a computer display, a mouse, a cursor, an artificial or prosthetic limb, a robot, or robotic device.

According to some embodiments, the computer/processor is further connected to same or different stimuli/manipulation system capable of delivering positive or negative feedback treatment to the brain organoid in culture.

According to some embodiments, the simulated behavior comprises psychiatric-related computer games evaluating social interaction, repetitive behavior, cognitive rigidity, and/or face recognition.

According to some embodiments, the psychiatric-related computer games comprise a positive or negative feedback.

In some embodiments, the positive or negative feedback treatment is same or different for the PD-derived brain organoid and the healthy brain organoid.

In some embodiments, each one of the simulations comprises random movement of two dots in 2D or 3D space.

In some embodiments, the determining a positive or negative feedback treatment based on the simulated behavior comprises positive feedback when the dots come close together and negative feedback when the dots go apart from each other.

According to some embodiments, the computer/processor connected to and/or running a simulator is configured to repeating steps (c) to (f) X times; wherein X is an integer between 1 and 100.

In some embodiments, X is an integer between 1-10000, 1-1000, or 1-100. Each possibility is a separate embodiment.

According to some embodiments, the computer/processor connected to and/or running a simulator is configured to compute an output score indicative of the learning-behavior response based on a change in the behavior of the brain organoid.

According to some embodiments, the computer/processor is configured to assess the severity of PD based on similarity to a predetermined learning-behavior response of a PD-derived brain organoid and/or predetermined learning-behavior response of a healthy brain organoid.

As used herein the term “a change in the behavior” refers to repeatedly considering the simulated behavior of a brain organoid in response to a determined and executed positive or negative feedback treatment, to compute a learning-behavior response and an output score indicative thereof.

According to some embodiments, the system is used for drug screening and/or evaluation of efficacy of treatment with a drug applied directly to the brain organoid in culture; according to some embodiments, the processor is further configured to obtain and take under consideration input data from brain organoid before and after being treated with the drug directly in culture; according to some embodiments, the system is further configured to compute a drug/treatment efficiency score based on a change in the learning-behavior response.

According to some embodiments, assessment of learning-behavior response associated with a psychiatric disorder (PD) comprises drug screening and/or evaluation of efficacy of treatment with a drug applied directly to the PD-derived brain organoid in culture; according to some embodiments, assessment of learning-behavior response associated with a psychiatric disorder (PD) comprises monitoring the progress of a suffering subject being treated with a drug; according to some embodiments, the drug for screening and/or evaluation is a known psychiatric/neurologic drug in medical use, or a potential drug for the treatment of PD.

Non-limiting examples of psychiatric/neurologic drugs comprise selective serotonin reuptake inhibitors (SSRIs), Selective serotonin and norepinephrine inhibitors (SNRIs), beta-blockers, stimulants, serotonergic drugs, tricyclic antidepressants, atypical antipsychotic agents, and lithium, alpha-2 agonists.

In some embodiments, computing an output score indicative of the learning-behavior response based on a change in the behavior of the brain organoid comprises an algorithm and/or AI (reinforcement learning algorithm) integrating the learning-behavior response score as well as additional biological data and/or physical data and/or molecular data and/or biochemical data and/or metabolitic data and/or information on origin of the subject.

According to some embodiments, there is provided a method for assessment of a learning-behavior response associated with a psychiatric disorder (PD), the method comprising: (i) obtaining a brain organoid; (ii) obtaining one or more signals of the brain organoid, wherein the obtaining of one or more signals comprises obtaining information indicative of neuronal function/activity derived from the one or more signals; (iii) computing/processing input data comprising the obtained information indicative of neuronal function/activity from the brain organoid; and generating a computer simulation of the brain-organoid behavior; (iv) determining a positive or negative feedback treatment based on the simulated behavior; (v) executing/delivering the determined positive or negative feedback treatment to the brain organoid using a stimuli/manipulation system; (vi) obtaining post-treatment input data from the brain organoid comprising information indicative of neuronal function/activity in response to the execution of the determined positive or negative feedback treatment; (vii) simulating a post-treatment behavior of the brain organoid data; (viii) repeating steps (iv) to (vii) X times; (ix) computing a degree of similarity of the computed learning-behavior response to a predetermined PD-derived learning-behavior response profile and/or to a predetermined healthy derived learning-behavior response profile and providing an output score indicative of the learning-behavior response based on the degree of similarity, thereby assessing the severity of PD.

According to some embodiments, the method for assessment of a learning-behavior response associated with a psychiatric disorder (PD) comprises obtaining a PD-derived brain organoid and a healthy brain organoid

According to some embodiments, there is provided a method for training a machine learning algorithm for assessment of a learning-behavior response associated with a psychiatric disorder (PD), the method comprising: (i) obtaining information indicative of neuronal function/activity from a plurality of PD-derived brain organoid and a plurality of healthy brain organoid; (ii) labeling the information indicative of neuronal function/activity of the plurality of PD-derived brain organoids as ‘PD-derived’ input data and the information indicative of neuronal function/activity of the plurality of healthy brain organoids as ‘healthy-derived’ input data (iii) computing/processing the input data comprising the labeled information; and generating a computer simulation indicative of a behavior of each of the PD-derived brain-organoids and a simulation indicative of a behavior of each of the healthy brain organoids; (iv) providing a positive or negative feedback treatment to each of the PD derived and healthy organoids, wherein the treatment is responsive to the simulated behavior; and obtaining post-treatment information indicative of neuronal function/activity of the plurality of PD-derived brain organoids and post-treatment information indicative of neuronal function/activity of the plurality of healthy brain organoids, in response to the provided feedback treatment; (v) labeling the plurality of post-treatment information indicative of neuronal function/activity of the plurality of PD-derived brain organoids as ‘PD-derived post-treatment’ input data and the plurality of information indicative of neuronal function/activity of the plurality of healthy brain organoids as ‘healthy-derived post-treatment’ input data; (vi) computing/processing the plurality of post-treatment input data comprising the labeled information; and simulate the behavior of the plurality of PD-derived brain-organoids and the behavior of the plurality of healthy brain organoid; (vii) computing a learning behavior response for each of the PD-derived brain-organoids and each of the healthy derived brain organoids, based on a change in the simulated behavior of the organoids in response to the treatment; (viii) computing an PD-derived brain organoid learning behavior response profile and a healthy organoid learning behavior response profile based on the learning behavior responses computed for each of the PD-derived and healthy-derived organoids.

According to some embodiments, the method for training a machine learning algorithm for assessment of a learning-behavior response associated with a psychiatric disorder (PD) further comprises validating the trained algorithm on a validation set comprising a plurality of unlabeled PD-derived behavior simulations and a plurality of unlabeled healthy derived behavior simulations obtained before and after feedback treatment.

According to some embodiments, the positive or negative feedback treatment is same or different for the PD-derived brain organoid and the healthy brain organoid.

According to some embodiments, each of the simulation for training a machine learning algorithm comprises random movement of two dots in 2D space.

According to some embodiments, the method for training a machine learning algorithm, wherein the determining of a positive or negative feedback treatment based on the simulated behavior comprises positive feedback when the dots come close together and negative feedback when the dots go apart from each other.

The following examples are presented in order to illustrate some embodiments of the invention more fully. They should in no way be construed, however, as limiting the broad scope of the invention. One skilled in the art can readily devise many variations and modifications of the principles disclosed herein without departing from the scope of the invention.

### EXAMPLES

### Materials and Method

Protocol for preparation of cortical organoids from hiPSCs—Cortical organoids were prepared based on Rosebrock N. et al., Nature Cell Biology 24, 981-995 (2022). Briefly, on day 0, hiPSC colonies were first incubated with 1 ml accutase up to 10 min until colonies detached. The colonies were then triturated until single cells were obtained. The accutase enzyme was neutralized by washing with hESC/KSR medium and centrifugation at 270 g for 5 min. Single cells were resuspended in 1 ml hESC/KSR medium containing FGF2 (4 ng/ml) and ROCK inhibitor (50 μM). The cells were enumerated and the volume of the hESC/KSR medium was adjusted along with FGF2 and ROCK inhibitor to a concentration of 9,000 cells per 150 μl. Suspended single cells were plated on a 96-well U-bottom low-attachment plate. The plate was inspected for cell aggregation and formation of embryoid bodies (EBs) on day 1. On day 2, half of the medium was aspirated without disturbing aggregates and 150 μl hESC/KSR medium was added to a total of 225 μl hESC medium along with the appropriate inhibitor molecule—SB-431542 (10 μM), LDN (200 nM) or XAV-939 (3.3 μM)—or a combination thereof. FGF2 and ROCK inhibitor were withdrawn once the EBs reached a size of approximately 350 μm. On day 4, 150 μl medium was removed and replaced with fresh 150 μl hESC/KSR medium along with the corresponding inhibitor molecules. On day 6, the organoids were transferred into a low-attachment 24-well plate along with N2 neural induction medium. Every alternate day, medium was aspirated and replaced by an equal volume of fresh N2 medium along with factors until day 11. On day 11, the organoids were embedded in 30 μl Matrigel droplets and incubated for 30 min in the incubator, after which they were transferred into a six-well low-attachment plate containing N2/NB medium along with 1% B27 without RA. On day 13, a medium change was made using the same medium from day 11. On day 15, the entire supernatant medium was removed and replaced with fresh medium containing N2/NB medium along with 1% B27 with RA; the organoid dishes were transferred onto an orbital shaker and the medium was changed daily. For long-term organoid culture, Matrigel (1%) was added directly to the culture medium and the medium was changed every 2 d.

The same protocol can be used for preparation of brain organoids from hESC.

### Example 1: Closed Loop and Open Loop System

The method underlying the herein disclosed system for assessment of a psychiatric disorder (PD) is based on determining a ‘behavior’ of a brain organoid in response to treatment/stimuli. The ‘behavior’ of the organoid corresponds to its neuronal function/activity in response to the treatment that was provided. The brain organoid may be a PD-derived brain organoid that is subjected to a compound or a medicament in order to determine its ability to affect the severity of PD of the organoid (i.e., evaluation of efficacy of a drug), or it can be an unknown/undetermined brain organoid that is being evaluated for having PD (i.e., diagnosis).

Determination of the brain organoid's behavior includes a comparison thereof with a prediction of behavior of a PD-derived organoid and/or a healthy organoid. The level of similarity to the predicted behavior enables evaluation of its PD severity (i.e., probability) and classification to a category of healthy or PD.

As can be seen in FIG. 1A system components, their structural and functional relations include: (1) a brain organoid in 2D/3D culture; (2) a sensor (multi-array electrode (MAE)) coupled to a recorder (recording head stage (RHS)) capable of detecting and recording signals from the brain organoid; (3) a micro-controller unit (MCU) configured to receive, integrate and/or transmit information/data indicative of neuronal function/activity derived from the signals; (4) Optionally FPGA (Field-Programmable Gate Array) or Equivalent Element, before, after or integrated in the MCU, responsible for high-speed parallel signal processing tasks, such as spike detection and classification, as well as the synchronization of stimulation triggers and/or, a computer/processor capable of determine response to stimuli/treatment sessions or an organoid behavior in response to stimuli/treatment sessions, and give instructions to provide a predetermined treatment (Open loop), or a feedback treatment (Closed loop), and computing an output at the end of the sessions; (5) optionally, a visualization component such as a screen/monitor/robot, or the like, connected to the processor/computer, and capable of presenting the computational simulation (i.e., the stimuli-response sessions/simulation) in a visual manner; (6) a stimuli/manipulation system connected to the processor/computer and capable of delivering treatment to the brain organoid in culture. (7) The system computes an output including computation of the overall behavior responses (Open loop), or a learning behavior responses (Closed loop), for PD, healthy or undetermined brain organoids derived from any developmental stage (e.g., prenatal, neonatal, mature baby, or adult); and (8) assesses probability of severity of PD based on similarity between the organoids behavior and classify accordingly (9) thereby providing a platform that can be utilized for drug screening and/or as a mean for personalized medicine aiming at evaluation/prediction of clinical success of treatment with a psychiatric drug, including a medicament for neurologic, neurodevelopmental, and neurodegenerative disease

As can be further seen in FIG. 1A, the system and methods provide two types of approaches for assessing severity of PD:

A first approach is the ‘open loop’ approach, which is based on a behavior in response to a predetermined treatment. In this approach the treatment is characterized by having “fixed” parameters, including its spatial pattern, intensity, duration, amplitude, frequency, concentration and/or temperature.

Since according to the ‘open loop’ the stimuli provided to the brain is repeated in a predetermined manner that does not consider the neural network response to former stimuli/stimuli sessions, the AI algorithm, which is a classification algorithm, repeatedly learns the behavioral response of the brain (to the functional cognitive assay) and classifies it accordingly. In accordance the AI algorithm is continuously reinforced, based on repetition in the determined brain-organoid behavior, to thereby improve the predicted behavior.

The classification algorithm is trained on brain-organoids behaviors of a plurality of healthy and/or PD derived brain organoids in response to the predetermined treatment/stimulus, wherein the training data is labeled according to one or more predetermined parameters of the treatment/stimulus.

A second approach is the ‘closed loop’ approach, which is based on a learning-behavior response that consider the change in the behavior (i.e., learning) of a brain organoid in response to positive or negative feedback treatment that is characterized by having “elastic” parameters including its overall spatial pattern, intensity, duration, amplitude, frequency, concentration and/or temperature.

Since according to the ‘closed loop’ the stimuli provided to the brain is a positive or negative feedback treatment that changes from session to session (elastic) based on the learning behavior of the brain organoid in response to previous treatments, the AI algorithm, which is a reinforcement learning algorithm, learns the ability of the brain organoid to learn (functional cognitive assays) and classifies it accordingly. In accordance the AI algorithm is continuously reinforced, based on the determined brain-organoid learning behavior response, to thereby improve the predicted learning behavior.

The reinforcement learning algorithm is trained on brain-organoids learning behaviors of a plurality of healthy and/or PD derived brain organoids in response to a positive or negative feedback treatment/stimulus, wherein the training data is labeled according to one or more changes in parameters of the treatment/stimulus.

In addition, the system is scalable. As can be seen in FIG. 1B the structure is scaled up by including a plurality of each unit, including a plurality of cultures brain organoids, plurality of RHS, plurality of MEA, and plurality of monitors.

### Example 2—Electrical Activity Recording from an ASD Derived Brain Organoid-On-a Chip, all in One System

A device was customized according to the design principles of the system for assessment of PD presented in hereinabove Example 1.

As can be seen in FIGS. 1C-1D the device is an ‘in-vitro lab’/‘organ-on-a chip’ designed to include at least: a brain organoid in 2D/3D culture and a sensor (multi-array electrode (MAE)) coupled to a recorder (recording head stage (RHS)). The device may further include a micro-controller unit (MCU), a processor and possibly a power source, making it an all-in-one device.

To test the device, a system integrating the device was established.

As can be seen in FIG. 1E-1F an ASD-derived cortical brain organoid (day 90) generated from cells of an ASD patient was plated and positioned on the plate holder in the device (FIG. 1E; I and enlarged FIG. 1F) which was then connected to a stimuli/manipulation system (FIG. 1E; II) capable of providing an electrophysiological stimulus to the brain organoid in culture, recording signals from the brain organoid, and transmitting the information/data indicative of neuronal function/activity derived from the signals directly to the computer (III), where the recording of the electrical activity detected from the organoid was visualized.

FIG. 2A shows a closer look on the interface between the ASD-derived brain organoid and the multi electrode array (MEA) belonging to the customized device.

As can be seen in FIG. 2A (left; ON; red dots) a sub-set of electrodes including 3 out of 59 ‘channels’ were then activated to send an electrophysiological stimulus in a spatiotemporal controlled manner including a desired pattern and parameters.

Next, a similar procedure was performed with a dissociated brain organoid in 2D culture.

The interface between the dissociated ASD-derived brain organoid in 2D culture and the multi electrode array (MEA) belonging to the customized device, can be seen in FIG. 2B.

The ASD-derived cells in the culture were dissociated from a brain organoid after its formation (126 days). The 3D brain organoid was processed/enzymatically digested to dissociated cells, by a commercial Papain dissociation system, and the cells were plated in 2D culture in the customized device.

### Example 3—Electrical Activity Recording from ASD Derived Brain Organoids Using Calcium Imaging

Next, recording of electrical activity from cortical brain organoids generated from cells of an ASD patient was performed using genetic reporters.

In this set up the organ-on-a chip costume device including the sensor (multi-array electrode (MAE)) coupled to a recorder (recording head stage (RHS)) used for detection and recording of electric signal from the 2D/3D culture of a brain organoid, was replaced with a imaging device coupled to a camera-a spinning disk confocal microscope equipped with incubator chamber for 2D/3D culture of the ASD-derived brain organoid and capable of detection of optic/light signal emitted from the organoid.

Calcium imaging was performed using genetic calcium indicator (GCaMP) 14 days after infection of the ASD derived brain organoids with AAV.

As can be seen in FIGS. 3A-3D green fluorescence signal indicative of successful imaging of calcium influx into neurons was detected from brain organoids in culture and recording of the calcium imaging/electrical activity was performed.

Green fluorescence was detected both from dissociated cortical brain organoid (day 126) expressing GCaMP8m calcium indicator (AAV9:hSyn1-GCaMP8m) (FIG. 3A), as well as from a small 3D clump (approx. 0.5 mm wide) of organoid tissue from partially dissociated organoid expressing the same GCaMP8m calcium indicator (FIG. 3B).

Recording of the signal was performed from another cortical brain organoid (Day86) generated from cells of an ASD patient and expressing GCaMP8m calcium indicator (AAV9: hSyn-jGCaMP7f) (FIG. 3C), and traces of signal indicative of electrical activity of the neurons were quantified (FIG. 3D).

### Example 4—Assessment of a Behavior Response Associated with a PD-Like Brain Organoid Using Open Loop Approach

A signal that is detected by the system provided herein, and results from spontaneous activity is not a meaningful signal that is indicative of true neuronal functioning that codes information. Therefore, in order to be able to distinguish between a network response related to PD or to healthy, one or more stimuli sessions are provided to the 3D organoid or the 2D neuronal culture, so an organoid behavior can be determined (whether PD like behavior or healthy like behavior) based on the response to the provided stimuli.

The following exemplifies the steps of the method for assessing PD related to the analyses of the recorded data, and determining the organoid behavior or the stem cell-derived 2D neuronal culture based on a simulation of the stimuli-response sessions that are visualized as a computer game, and further classifying the behavior (whether PD like behavior or healthy like behavior) or scoring the result as a likelihood for certain severity of PD.

The stem cell-derived 2D neuronal culture of the example were generated and stimulated, and their network response was recorded.

The visual simulation presented in FIG. 4A exemplifies stem cell-derived 2D neuronal culture behavior that was determined by the provided system based on the network response.

The visual simulation presented in FIG. 4B is a theoretical example of how the behavior of the stem cell-derived 2D neuronal culture can be visualized after determining it based on the network response.

A non-limiting example of a type of analysis that underly determining the network/organoid behavior is exemplified in FIG. 4D.

In this regard, reference is also made to the method for training an AI algorithm for determining organoids behavior.

Stem cell-derived 2D neuronal culture were generated from cells derived from a healthy donor, at least with respect to having PD, and were divided into two groups: a control group of neuronal culture, and a group of neuronal culture that were subjected to a perturbation by competitive antagonist at GABA type A receptors that models/mimics PD (PD-like perturbation).

The two groups (FIG. 4A; two ‘players’), healthy control neuronal culture and PD-like perturb neuronal culture were cultured in the system for assessment of PD and were subjected to predetermined/‘fixed’ treatment/stimuli.

The processor of the system was further connected to a visualization component presenting a functional cognitive computer simulation/assay that examined the behavior/response of the neuronal culture (the two ‘players’) to the predetermined/‘fixed’ treatment/stimuli.

For example, a predetermined stimuli (‘fixed’ treatment) presented as coin in the computer game, may include one or more repetitive sessions having same pattern including for example 2 ‘short’ stimulus having high amplitude at the right region of the organoid, followed by a longer stimulus having low amplitude at left region of the organoid.

The processor runs an AI algorithm, which that was trained on neuronal culture behaviors of a plurality of healthy and/or PD derived neuronal culture in response to the predetermined treatment/stimulus described above, and the training data was labeled according to one or more of the predetermined parameters of the treatment/stimulus (e.g., the spatial orientation, amplitude, frequency, duration etc.,).

The AI algorithm repeatedly learned the behavioral response of the healthy control neuronal cultures and the PD-like perturb neuronal culture in response to the functional cognitive assay, i.e., performance of the ‘players’, compared it with the training data, classified it according to its level of similarity, and scored the probability that the ‘player’ is healthy.

Advantageously and surprisingly, as can be seen in FIG. 4A by the end of the game the PD-like perturb neuronal culture (Right; 3 green squares) was evaluated as having only 60% chance of being healthy as compared to the control neuronal culture which was scored as having 100% chance of being healthy (Left; 5 green squares).

FIG. 4B presents another example of computer simulation/assay that is performed to evaluate cognitive abilities, and can be used as part of both open and closed modes.

Here, a dot movement in 2D space represents a neuronal culture in response to a predetermined stimulus, whereas a dot that moves in a periodically manner through space (i.e., in the same pattern) is classified as ASD-derived, while a dot that moves more randomly through space is classified as healthy.

### Example 5—Assessment of a Learning-Behavior Response Associated with a ASD-Derived Brain Organoid Using a Closed Loop Approach for Assaying Social Interaction Simulation

A signal that is detected by the system provided herein, and results from spontaneous activity is not a meaningful signal that is indicative of true neuronal functioning that codes information. Therefore, in order to be able to distinguish between a network response related to PD or to healthy, one or more stimuli sessions are provided to the 3D organoid or the 2D neuronal culture, so an organoid behavior can be determined (whether PD like behavior or healthy like behavior) based on the response to the provided stimuli.

The following exemplifies the steps of the method for assessing PD related to the analyses of the recorded data, and determining the organoid behavior based on a simulation of the stimuli-response sessions that are visualized as a computer game, and further classifying the behavior (whether PD like behavior or healthy like behavior) or scoring the result as a likelihood for certain severity of PD.

The visual simulation that is presented in FIG. 4C are a theoretical example of how the organoid behavior can be visualized after determining it based on the network response.

A non-limiting example of a type of analysis that underly determining the network/organoid behavior is exemplified in FIG. 4D.

In this regard, reference is also made to the method for training an AI algorithm for determining organoids behavior.

The processor is further connected to a visualization component presenting a social interaction computer simulation/assay that examined the capacity of the brain organoid to learn social interaction abilities by determining the learning-behavior of the organoid in response to previous treatments/sessions where a positive or negative feedback treatment was provided, i.e., “elastic” parameters including stimulus spatial pattern, intensity, duration, amplitude, frequency).

The AI algorithm, which is a reinforcement learning algorithm, learns/determines the ability of the brain organoid to learn from session to session where a positive or negative feedback treatment was provided and classify it accordingly.

FIG. 4C provides an example of how such a ‘game’ is conducted. A healthy organoid (presented as dots in the computer game) gets closer and closer as they receive positive feedback, while ASD-derived organoids randomly drift in space as they receive negative feedback.

More specifically, during the session the behavior of the ASD-derived brain organoid is simulated using a random movement in a 2D space of two dots, each is simulating the behavior of a different ASD-derived brain organoid generated and obtained from the same subject. FIG. 4C shows that when the dots come closer to each other interaction occurs and a positive feedback treatment is determined/executed, but when the dots move apart from each other and there is no interaction a negative feedback treatment is determined/executed. A learning-behavior response is computed based on the change in the post-treatment behavior of the ASD-derived brain organoid.

The reinforcement learning algorithm is trained on brain-organoids learning behaviors of a plurality of healthy and/or PD derived brain organoids in response to a positive or negative feedback treatment/stimulus, and the training data is labeled according to one or more changes in parameters of the treatment/stimulus.

Advantageously and surprisingly the simulation demonstrates that by the end of the session the dots of the ASD-derived brain organoid are positioned far apart from each other in comparison to dots representing a simulated behavior of a healthy-brain organoid.

### Example 6—Compound Screening in-Culture and Personalized Medicine

In some embodiments, PD-derived brain organoids are used for compound screening in-culture (e.g., drug discovery) and for personalized evaluation of treatment with a medicament, based on its efficacy in-culture or in-vivo.

Advantageously, for drug screening a plurality of potential compounds is added to a plurality of PD-derived brain organoids in culture, and the effect of the compounds is evaluated by the system for assessing PD, using an open or closed methodology, compared with non-treated PD-derived brain organoids.

Advantageously, for personalized evaluation of treatment efficacy with a psychiatric/neurologic/neurodevelopmental medicament, the medicament is added to PD-derived brain organoids generated from the suffering patient and the efficacy of the medicament is evaluated in culture by the system for assessing PD, using an open or closed methodology, compared with non-treated PD-derived brain organoids.

Furthermore, personalized evaluation of treatment efficacy may be performed after the suffering patient itself was treated. Here the treatment may include, for example, but is not necessarily limited to a psychiatric/neurologic/neurodevelopmental medicament, or to genetic or electromagnetic treatment A suffering patient is treated/administered with a therapeutically effective amount of psychiatric/neurologic/neurodevelopmental medicament, PD-derived brain organoids are generated from the suffering patient before and after the treatment with the medicament, and the efficacy of the treatment is evaluated in culture by the system for assessing PD, using an open or closed methodology, by comparing the brain organoids generated from the suffering patient before and after the treatment.

### Example 7—Generation and Molecular Characterization of Cortical Brain Organoids Generated from Prenatal Cells

The following is a non-limiting example for brain organoid generation.

Cortical brain organoids were generated from human primary prenatal cells according to the protocol for preparation of cortical organoids from hiPSCs, and were characterized in-situ using immunostaining for spatial expression of specific developmental markers.

In addition, a prenatal derived iPSC line was established to provide convenient and consistent resource for prenatal-derived healthy organoids.

Primary neonatal cells-Human Amniotic Epithelial Cells (HAEpiC)-were isolated from an amniotic fluid sample collected from a fetus by amniotic fluid test (Amniocentesis), and were used to generate iPSC line followed by generation of 3D self-assembled structures.

Developmental differences, including in morphology/structure and in gene expression, between the primary neonatal cells, the iPSC, and the 3D organoids derived therefrom, were examined.

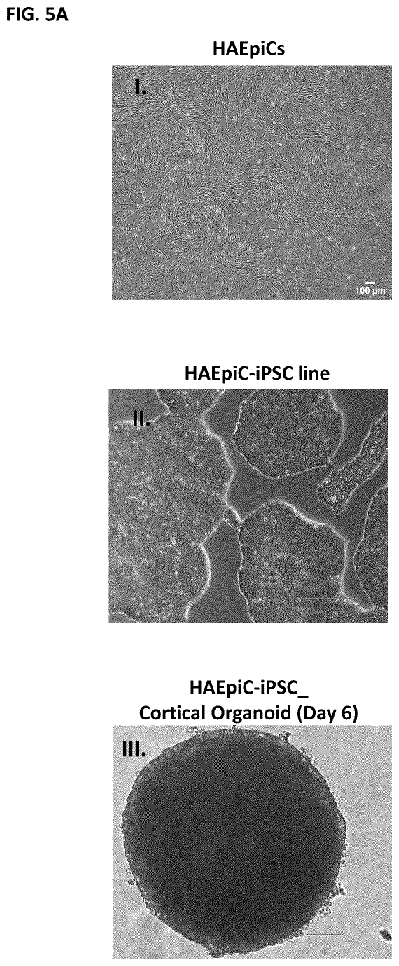

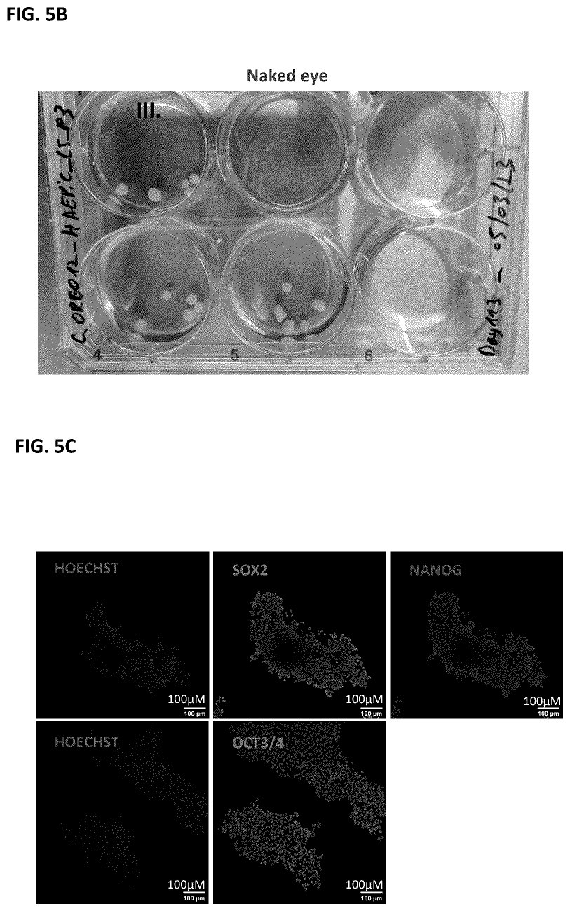

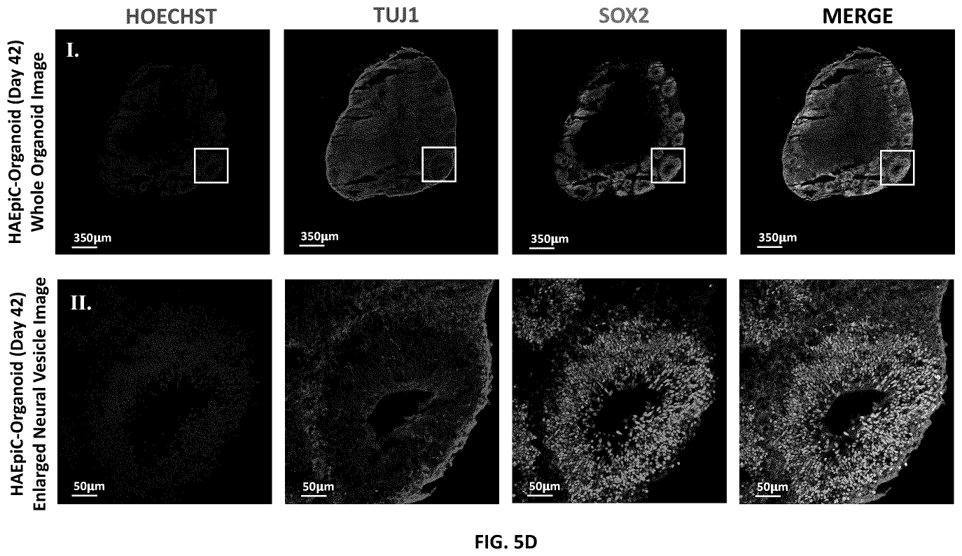

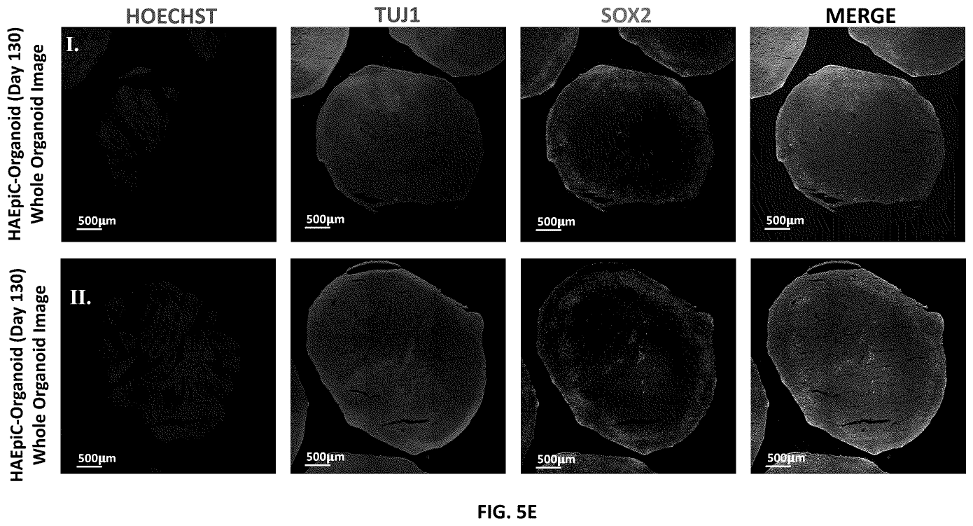

As seen in FIG. 5A the main stages of generating cortical brain organoids include generation of iPSC from the primary neonatal cells followed by development and growth of 3D at least partially self-assembled structures, i.e., cortical organoids.

As an alternative, the starting point may be hESC instead of iPSC.

Exemplified herein are human amniotic epithelial cells (HAEpiCs) isolated from amniotic fluid sample collected from a fetus by amniotic fluid test (Amniocentesis). The obtained cells were reprogrammed into induced pluripotent stem cells (iPSCs) and a line derived from the HAEpiCs (HAEpiC-iPSC line) was established, to provide a source based on which populations of 3D cortical organoids, visible to the naked eye, were generated and grown at least for 130 days (FIG. 5B). timeline is according to the same protocol for preparing organoids.

To characterize the organoid for spatial expression of specific developmental markers, whole mount immunostaining was performed to cells of the HAEpiC-iPSC line, as well as for the 3D generated organoids (FIGS. 5C-5E).

First, as seen in FIG. 5C, immunostaining of cells of the HAEpiC-iPSC line showed that the cells express pluripotent markers including SOX2, NANOG, and OCT3/4, suggesting these cells are undifferentiated and have the capacity for self-renewal.

However, during organogenesis, altered expression of these transcription factors influences the stem cells to lose their pluripotency and turn toward a lineage selection.

To assess lineage selection of HAEpiC-cortical organoids, the 3D brains were subjected to whole mount immunostaining for spatial expression analysis of neuronal marker (TUJ1) and neural stem cell markers (SOX2), at day 42, and at day 130.

As can be seen in FIG. 5D, neural vesicle/rosette structures were observed at 42 days old organoids (top; I.). The enlarged image of the neural vesicle/rosette displays neural stem cells (SOX2) around the ventricle and neurons (TUJ1) surrounding the neural stem cells (bottom; II.).

In addition, as can be seen in FIG. 5E expression of TUJ1 and SOX2 was detected in 130 days old HAEpiC-Cortical Organoids, thereby demonstrating the presence of neurons.

In summary, cortical brain organoids were self-assembled into an organized 3D structure having neural vesicle/rosette motifs/structures including both differentiated neurons and neural stem cells arranged in a coordinated manner. The organoids were generated from primary amniotic prenatal cells, and a prenatal derived iPSC line was established therefrom.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-54. (canceled)

55. A system for assessment of a psychiatric disorder (PD), the system comprising:
(i) a brain organoid and/or stem cell-derived 2D neuronal culture;
(ii) a stimuli system capable of delivering stimuli to the brain organoid and/or the neuronal culture;
(iii) a sensor coupled to a recorder capable of detecting and recording one or more signals indicative of neuronal function/activity of the brain organoid and/or the neuronal culture;
(iv) a micro-controller unit (MCU) configured to receive, integrate and/or transmit data of the one or more signals; and
(v) a computer/processor configured to:
a. send instructions to the stimuli system to provide one or more stimuli sessions, each session comprising a stimuli provided to the brain organoid and/or the neuronal culture;
b. obtain data recorded in response to the one or more stimuli sessions, the data indicative of neuronal function/activity of the brain organoid and/or the neuronal culture;
c. determine a brain-organoid behavior and/or a neuronal culture behavior based on the recorded data; and
d. apply an AI algorithm on the brain-organoids behavior and/or the neuronal culture behavior to thereby classify the brain organoid based on a degree of similarity of the determined brain-organoid behavior to a predicted behavior of a PD-derived brain organoid and/or a heathy organoid, and/or to thereby classify the neuronal culture based on a degree of similarity of the determined neuronal culture behavior to a predicted behavior of a PD-derived neuronal culture and/or a heathy culture.

56. The system of claim 55, wherein the AI algorithm is trained on brain-organoids behaviors of a plurality of healthy and/or PD derived brain organoids, and/or trained on neuronal culture behaviors of a plurality of healthy and/or PD derived neuronal cultures, in response to the predetermined stimulus, wherein the training data is labeled according to one or more parameters of the stimulus.

57. The system of claim 55, wherein the AI algorithm is continuously reinforced, based on the determined brain-organoid behavior and/or based on the determined neuronal culture behavior, to thereby improve the predicted behavior.

58. The system of claim 55, comprising a closed loop, in which the stimulus provided to the brain organoid and/or to the neuronal culture is determined according to the determined brain-organoid behavior and/or neuronal culture behavior.

59. The system of claim 55, wherein classifying the brain organoid and/or neuronal culture is based on a degree of similarity of the learning-behavior response to a predicted learning-behavior response of a PD-derived brain organoid and/or of a heathy organoid and/or to a predicted learning-behavior response of a PD-derived neuronal culture and/or heathy neuronal culture.

60. The system of claim 55, further comprising a visualization component presenting a visual simulation representative of the determined organoid behavior and/or of the determined neuronal culture behavior, and wherein the visual simulation comprises a computer game evaluating cognitive abilities selected from one or more of: memory, cognitive rigidity, motivation, repetitive behavior, attention, social interaction, processing speed, executive function, numerical abilities, and/or facial expression, or any combination thereof.

61. The system of claim 55, further comprising assessing the severity of PD based on the similarity.

62. The system of claim 55, wherein the processor is further configured to repeat steps a-c on the brain organoid and/or on the neuronal culture after treatment thereof with a neurological neurodevelopmental and/or neurodegenerative medicament, or any combination thereof and to determine an efficacy of the treatment.

63. The system of claim 55, wherein the brain organoid is an undetermined brain organoid having unknown severity of PD, and/or wherein the neuronal culture is an undetermined neuronal culture having unknown severity of PD.

64. The system of claim 55, wherein the obtained brain organoid comprises 3D brain organoid in culture.

65. The system of claim 55, wherein the sensor comprises one or more multi-array electrode (MAE) coupled to one or more recording head stage (RHS).

66. The system of claim 55, wherein the stimuli/treatment provided by stimuli system comprises electrophysiological stimuli.

67. The system of claim 55, wherein the data indicative of the neuronal function/activity comprises spatiotemporal propagation including spatial distribution and time after stimulation, intensity, frequency, and amplitude of the detected signal, or any combination thereof.

68. The system of claim 55, wherein the PD is Autistic Spectrum Disorder (ASD).

69. A method for assessment of a psychiatric disorder (PD), the method comprising:
a. obtaining a brain organoid and/or obtaining stem cell-derived 2D neuronal culture;
b. providing one or more stimuli sessions, each session comprising a stimuli provided to the brain organoid and/or to the neuronal culture;
c. obtaining data recorded in response to the one or more treatment/stimuli sessions, the data is indicative of neuronal function/activity of the brain organoid and/or of neuronal function/activity of the neuronal culture;
d. determining a brain-organoids behavior, and/or determining a neuronal culture behavior, based on the recorded data; and
e. applying an AI algorithm on the brain-organoids behavior for classifying the brain organoid based on a degree of similarity of the determined brain-organoid behavior to a predicted behavior of a PD-derived brain organoid and/or a heathy organoid, and/or on the neuronal culture behavior for classifying the neuronal culture based on a degree of similarity of the determined neuronal culture behavior to a predicted behavior of a PD-derived neuronal culture and/or a heathy neuronal culture.

70. The method of claim 69, wherein the AI algorithm is trained on brain-organoids behaviors of a plurality of healthy and/or PD derived brain organoids, on neuronal culture behaviors of a plurality of healthy and/or PD derived neuronal culture, in response to the predetermined treatment/stimulus, wherein the training data is labeled according to one or more predetermined parameters of the treatment/stimulus.

71. The method of claim 69, wherein the AI algorithm is a trained on brain-organoids behaviors of a plurality of healthy and/or PD derived brain organoids, on neuronal culture behaviors of a plurality of healthy and/or PD derived neuronal culture, wherein the training data is labeled according to changes in one or more parameters of the treatment/stimulus.

72. The method of claim 69, wherein classifying the brain organoid, and/or the neuronal culture, is based on a degree of similarity of the learning-behavior response to a predicted learning-behavior response of a PD-derived brain organoid and/or of a heathy organoid, and/or to a predicted learning-behavior response of a PD-derived neuronal culture and/or of a heathy neuronal culture.

73. The method of claim 69, further comprising assessing the severity of PD based on the similarity.

74. The method of claim 69, wherein the PD is Autistic Spectrum Disorder (ASD)
